(19) United States
(12) Patent Application Publication
(10) Pub. No.: US 2005/0084916 A1
(43) Pub. Date: Apr. 21, 2005

(54) Identification of the IkappaBNS protein and its products

(75) Inventors: Ellis L. Reinherz, Lincoln, MA (US); Linda K. Clayton, Jamaica Plain, MA (US); Emma Fiorini, Cambridge, MA (US); Pedro A. Reche, Boston, MA (US); Ingo Schmitz, Kaarst (DE)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Dana-Farber Cancer Institute, Inc., Boston, MA (US)

(21) Appl. No.: 10/783,994

(22) Filed: Feb. 20, 2004

Related U.S. Application Data
(63) Continuation of application No. PCT/US02/08288, filed on Mar. 14, 2002.
(60) Provisional application No. 60/314,046, filed on Aug. 22, 2001.
(60) Provisional application No. 60/322,993, filed on Sep. 18, 2001.

(51) Int. Cl. G01N 33/574; C07H 21/04; C12N 9/64

(52) U.S. Cl. 435/7.23; 536/23.2; 435/69.1; 435/226; 435/320.1; 435/325

(57) ABSTRACT

The identification of a novel gene, termed IκBNS, regulated during negative selection of immature CD4⁺CD8⁺double positive (DP) thymocytes in mice, and the human homolog of the gene, are described, along with the gene products produced by these genes. Methods of using the disclosed genes and gene products are also described.

### RELATED APPLICATIONS

This application is a continuation of International Application No. PCT/US02/08288, which designated the United States and was filed 14 Mar. 2002, published in English, which claims the benefit of U.S. Provisional Application No. 60/314,046, filed on Aug. 22, 2001, and U.S. Provisional Application No. 60/322,993, filed on Sep. 18, 2001.

The entire teachings of the above applications are incorporated herein by reference.

### GOVERNMENT SUPPORT

The invention was supported in part, by the National Institutes of Health grant numbers AI45022 and AI19807. The Government has certain rights in the invention.

### BACKGROUND OF THE INVENTION

Apoptosis is a morphologically stereotyped form of programmed cell death utilized by metazoan organisms during normal development as well as for homeostasis (Kerr et al., 1972; Horvitz et al., 1982; Steller, 1995; Vaux and Korsmeyer, 1999). In T lymphoid development, αβ T cell receptors (TCR) are generated by a stochastic process of rearrangement of variable gene segments. Through combinatorial diversity, billions of different TCRs are generated ensuring protection against a myriad of infectious agents without a requirement to encode all TCR specificities in the germline (Davis and Bjorkman, 1988). One potential hazard of this diversity-generating mechanism is the creation of self-reactive TCRs. Those TCRs interacting too strongly with self-proteins bound to MHC molecules are deleted (negative selection) and those weakly reactive are preserved (positive selection). However, the processes influencing the selection of immune reactive T cells for development are poorly understood.

### SUMMARY OF THE INVENTION

Work described herein details the identification of a novel gene regulated during the negative selection of immature CD4⁺ CD8⁺ double positive (DP) thymocytes. A systematic method of analyzing differentially expressed genes using TCR transgenic mice has resulted in the discovery of a negative selection gene, termed IκBNS, in the mouse and its homolog in the human. The IκBNS protein contains seven ankyrin repeats and it is homologous to known IκB family members but lacks ubiquitination-based degradation signals. In class I and class II MHC-restricted TCR transgenic mice, transcription of IκBNS is stimulated by peptides that trigger negative selection but not by those inducing positive selection (i.e., survival) or by non-selecting peptides. IκBNS blocks transcription from NF-κB reporters, alters NF-κB electrophoretic mobility shifts and interacts with NF-κB proteins in thymic nuclear lysates following TCR stimulation.

The present invention relates to an isolated nucleic acid molecule consisting of a nucleic acid sequence selected from the group consisting of SEQ ID NOS: 1 and 3, the complement of SEQ ID NOS: 1 and 3 and nucleic acid sequences that encode SEQ ID NOS: 2 and 4. The invention also relates to an isolated nucleic acid molecule consisting of exons I to XIII of SEQ ID NO: 1 in a contiguous sequence, or combinations or permutations of exons I to XIII of SEQ ID NO: 1 in a contiguous sequence. The invention further relates to an isolated portion of SEQ ID NOS: 1 and 3, an isolated portion of the complement of SEQ ID NOS: 1 and 3, and an isolated portion of nucleic acid sequences that encode SEQ ID NOS: 2 and 4, wherein such portion is of sufficient length to distinctly characterize the sequence. For example, the isolated portion can be from about 10 to 25 nucleotides in length, from about 25 to 40 nucleotides in length, from about 40 to 200 nucleotides in length or from about 200 to 500 nucleotides in length. In a preferred embodiment, the portion is greater than 500 nucleotides in length.

The nucleic acid sequences of the invention can be DNA or RNA, single stranded or double stranded, sense or anti-sense, coding or non-coding sequence.

The present invention also relates to an isolated nucleic acid molecule comprising a nucleic acid sequence selected from the group consisting of SEQ ID NO: 3, the complement of SEQ ID NO: 3, a nucleic acid sequence that encodes SEQ ID NOS: 2 and 4, and to an isolated portion of SEQ ID NO: 3, an isolated portion of the complement of SEQ ID NO: 3, and an isolated portion of a nucleic acid sequences that encode SEQ ID NOS: 2 and 4, wherein such portion is of sufficient length to distinctly characterize the sequence. The present invention further relates to an isolated nucleic acid molecule comprising a nucleic acid sequence that encodes exons I to XIII of SEQ ID NO: 1 in a contiguous sequence, or combinations or permutations of exons I to XIII of SEQ ID NO: 1 in a contiguous sequence.

The invention further relates to an isolated nucleic acid molecule comprising a nucleic acid sequence that hybridizes under high stringency conditions to a nucleic acid sequence selected from the group consisting of SEQ ID NOS: 1 and 3, the complement of SEQ ID NOS: 1 and 3, and nucleic acid sequences that encode SEQ ID NOS: 2 and 4. The invention further relates to an isolated nucleic acid molecule hybridizing under high stringency conditions to a nucleotide sequence selected from the group consisting of an isolated portion of SEQ ID NOS: 1 and 3, an isolated portion of the complement of SEQ ID NOS: 1 and 3, and an isolated portion of nucleic acid sequences that encode SEQ ID NOS: 2 and 4, wherein such portion is of sufficient length to distinctly characterize the sequence. In a further embodiment, a nucleic acid molecule of the invention comprises a nucleotide sequence which is greater than about 75 percent, more preferably greater than about 80 percent, and even more preferably greater than about 90 percent, identical to a nucleotide sequence selected from the group consisting of SEQ ID NOS: 1 and 3, the complement of SEQ ID NOS: 1 and 3, nucleic acid sequences that encode SEQ ID NOS: 2 and 4, an isolated portion of SEQ ID NOS: 1 and 3, an isolated portion of the complement of SEQ ID NOS: 1 and 3, and an isolated portion of nucleic acid sequences that encode SEQ ID NOS: 2 and 4, wherein such portion is of sufficient length to distinctly characterize the sequence.

The invention also relates to a probe comprising a nucleic acid sequence that hybridizes under high stringency conditions to a nucleic acid sequence selected from the group consisting of SEQ ID NOS: 1 and 3, the complement of SEQ ID NOS: 1 and 3, nucleic acid sequences that encode SEQ ID NO: 2 and 4, a portion of SEQ ID NO: 1 and 3, and a portion of the complement of SEQ ID NOS: 1 and 3, wherein such portion is of sufficient length to distinctly characterize the sequence. In preferred embodiments, the fragment is useful as a probe, or primer and is at least 15, more preferably at least 18, and even more preferably 20-25, 30, 50, 100, 200 or more nucleotides in length.

The invention also relates to DNA constructs comprising a nucleic acid molecule as described herein, operatively linked to a regulatory sequence, as well as to recombinant host cells, such as bacterial cells, fungal cells, plants cells, insect cells, avian cells, amphibian cells and mammalian cells, comprising the nucleic acid molecules described herein, preferably operatively linked to a regulatory sequence. The invention further relates to methods for preparing proteins and polypeptides encoded by the isolated nucleic acid molecules described herein, comprising culturing the recombinant host cell, which comprises an isolated nucleic acid molecule to the invention operatively linked to a regulatory sequence.

The invention further relates to isolated proteins and polypeptides, or functional portion thereof, encoded by the nucleic acid molecules of the invention. In particular, the invention related to a protein or polypeptide having an amino acid sequence selected from the group consisting of SEQ ID NOS: 2 and 4. In addition the invention relates to proteins or polypeptides of the invention fused to another component, including but not limited to a GST protein, FLAG-tags, hemophilus influenza hemaglutinin tag or tags to direct cellular localization.

The invention also pertains to antibodies, including monoclonal and polyclonal antibodies, or antigen-binding fragments thereof, that selectively bind to the proteins and polypeptides of the invention, or portions thereof. Furthermore, the invention relates to a method for assaying the presence or absence of the protein or polypeptide, or portion of the protein or polypeptide, of the invention (e.g. encoded by a nucleic acid of the invention) in a sample, for example in a tissue sample, comprising contacting said sample with an agent (e.g. an antibody) which specifically binds to the encoded protein or polypeptide and detecting the formation of a complex between the protein or polypeptide and the agent.

Furthermore, the invention relates to a method to identify protein-interaction partners that interact with protein or polypeptide of the invention, or functional portion thereof, comprising the steps of contacting the protein or polypeptide with the agent to be tested and assaying for presence or absence of complex formation between the polypeptide and agent.

The invention further relates to a method of screening for an agent that is an agonist, mimic or antagonist or the proteins or polypeptides of the invention comprising the steps of contacting the protein or polypeptide, or functional fragment thereof, with the agent to be tested, and determining the level of activity of the polypeptide in the presence of the agent. Comparison of the activity of the protein or polypeptide in the presence of the agent with the level of activity of the protein or polypeptide in the absence of the agent determines if the agent is capable of altering the activity of the polypeptide. In a preferred embodiment, the activity of the protein or polypeptide is the modulation of NFκB-induced gene expression.

Non-human transgenic animals are also included in this invention. Such animals are transgenic for a nucleic acid sequence selected for the nucleic acid sequences of the invention, or nucleic acid sequences encoding a polypeptide of the invention. Furthermore, the non-human transgenic animals can be used to identify agents that alters the activity of a polypeptide molecule of the invention, or functional fragments thereof, comprising exposing a transgenic animal to the agent to be tested and determining the level of activity of the polypeptide or functional fragment thereof, wherein increased activity indicates that the agent is an agonist. In contrast, when decreased activity of the polypeptide, or functional fragment thereof, is detected, the agent is an antagonist. Preferably, the activity of the protein or polypeptide is modulation of NFκB-induced gene expression or binding to NFκB. In another embodiment of the invention, non-human transgenic animals that have all, or a fragment, of a nucleic acid molecule of the invention deleted are also envisioned.

The invention further relates to a method to identify gene targets of the polypeptides of the invention, comprising comparing the gene expression in a non-human transgenic animal that express a polypeptide of the invention with a non-human transgenic animal that does not express the same polypeptide, wherein genes identified to be modulated in association with expression of said polypeptide, and which are not modulated when the same polypeptide is not expressed, are potential targets of the polypeptide.

The invention also relates to a method to identify gene targets of the polypeptides of the invention, or functional fragments thereof, comprising comparing the gene expression in a recombinant host cell containing a polypeptide of the present invention with gene expression of a host cell that does not express the polypeptide, wherein genes identified to be specifically expressed in association with expression of the polypeptide, are potential targets of the polypeptide.

Furthermore, the invention relates to a method of treatment of an individual having a disorder comprising administering a therapeutically-effective amount of an agent that modulates the activity of the polypeptide of the invention. In a preferred embodiment, the disorders include, but are not limited to an autoimmune disease and the agent that modulates the activity of the polypeptide is an agonist. In another preferred embodiment, the disorders include, but are not limited to cancer, malaria, tuberculosis, and HIV infection and the agent that modulates the activity of the polypeptide is an antagonist. Administration of the agent can be performed by any suitable method, including but not limited to orally, intravenously, intramuscularly, subcutaneously, topically, rectally, or by inhalation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the scheme of N15tg RAG-2^{-/-} DP thymocyte interactions with selecting versus nonselecting thymic stromal cells. In the presence of H-2^{b} class I MHC bearing stromal elements, the exogenously administered VSV8 peptide binds K^{b} molecules forming a pMHC complex (step 1). DP thymocytes then recognize this pMHC complex in the N15 TCRtg RAG-2^{-/-} H-2^{b} mice via their TCR (step 2). Immune recognition triggers a TCR signal (step 3) resulting in the induction by the specific VSV8/K^{b} pMHC ligand of negative selection in those thymocytes (step 4). In contrast, in the N15 TCRtg RAG-2^{-/-} H-2^{d} mice, there is no N15 TCR recognition of endogenous peptides bound to K^{d} or other H-2^{d} molecules. Hence, no TCR signal occurs and no induction of negative selection. The clonotypic TCR αβ heterodimer and associated CD3εγ, CD3εδ and ζζ dimers are schematically represented. RNAs were derived from these two cell sorter-purified DP thymocyte populations and used for RDA.

FIG. 2 shows the multiple sequence alignment with IκBNS (SEQ ID NO: 4). The full sequence of IκBNS is shown. Numbering refers to IκBNS. The ankyrin domains of IκBNS are boxed and labeled from A to G. Secondary structure (ss) predictions for IκBNS are shown above the alignment with the inner helix of the ankyrin repeat core shown in gray, and the outer in blue. Secondary structure motifs of IκBα were obtained from pdb 1NFI (Jacobs and Harrison, 1998) and are shown below the alignment. Sequences were aligned using the program ClustaIX (Thompson et al., 1997), and the secondary structure prediction of IκBNS was determined using PSI-PRED (Jones, 1999). A dendrogram of the figure is displayed with IκBα as the root and was derived using the Neighborjoining method (Saitou and Nei, 1987). Amino acid positions with identities or similarities in 5 or more of the 6 proteins aligned are highlighted in black with yellow letters. Amino acid positions with identities or similarities in four or more of the 6 proteins are highlighted in gray. For this analysis, V/L/I, S/T, N/Q, D/E, K/R and W/F are considered equal. Residues shown are: for human IκBα aa 66-287, for human Bcl-3 aa 31-278, for murine IκBζ aa 292-629, for human p105 aa 522-756 and for murine p100 aa 467-705.

FIG. 3A is an RNA blot analysis of IκBNS expression in mouse tissues. A mouse multiple tissue Northern was probed with random-prime labeled IκBNS (upper panel) or β-actin control probe (lower panel). Each lane contains 2 μg of polyA+ mRNA. The IκBNS blot was exposed for 4 days; the β-actin blot was exposed for 4 h. The position of a 2.0 kb marker is indicated.

FIG. 3B is an RNA blot analysis of IκBNS expression in thymii of variously treated animals. 10 μg of total thymic RNA was run in each lane. RNA was isolated from the thymus of untreated N15tg RAG-2^{-/-} H-2^{d} animals, N15tg RAG-2^{-/-} H-2^{b} animals 1 h after i.v. injection of 24 μg VSV8, untreated TCRCyt5CC7-I-RAG-2^{-/-} H-2^{a} animals, TCRCyt5CC7-I-RAG-2^{-/-} H-2^{a} animals 1 h after i.v. injection of 24 μg PCC peptide, untreated C57BL/6 animals and C57BL/6 animals 1 and 2 h after injection of 0.5 mg dexamethasone or 500 rads whole body γ-irradiation.

FIG. 3C is an RNA blot analysis of IκBNS expression in fetal thymic organ cultures (FTOC). 4 μg of total thymic RNA isolated from N15tg RAG-2^{-/-} H-2^{b} FTOC was run in each lane. RNAs were prepared from FTOC after 2 h incubation with 10 μM of the indicated peptides. RNAs were run on agarose/formaldehyde gels, transferred to nylon membranes and probed first with randomprimed labeled IκBNS (upper panel) and then with random-primed labeled GAPDH (lower panel) as a loading control. The position of the 18S ribosomal RNA band is indicated.

FIG. 4 demonstrates that IκBNS inhibits NF-κB-induced expression of luciferase in transfected Cos-7 cells. Cos-7 cells were cotransfected with pRL-null and (kB3) NF-κB/luciferase constructs plus empty vector or, alternatively, cotransfected with pRL-null and (kB3) NF-κB/luciferase constructs plus IκBNS or IκBα constructs. 24 h after transfection, PMA was added to induce NF-κB activity. After 7 h, Cos-7 cells were lysed and luciferase activity determined. The relative light units (RLU) are shown ×10⁻³ on the ordinate. One representative experiment out of 5 is shown.

FIG. 5 shows the inhibition of NF-κB EMSAs by IκBNS. Nuclear thymic lysates were prepared from N15tg RAG-2^{-/-} H-2^{b} animals untreated (Control) or 1 h after i.v. injection of 24 μg VSV8 (VSV8) and assayed by EMSA with no additions (Control and −), 1 μg of GST protein, 1 μg of GST-IκBNS, or 1 μg GST-IκBα. Also added were a 100 fold excess of cold NF-κB probe or cold AP-1 probe. The upper panel uses an NF-κB probe and the lower panel an AP-1 probe. A probe only lane (Probe) without lysate is shown.

FIG. 6A is an analysis of the interaction of GST-IκBNS or GST-IκBα with NF-κB components in vivo and in vitro. Nuclear and cytosolic thymic lysates were prepared from uninjected N15 TCRtg RAG-2^{-/-} H-2^{b} mice or mice 10, 30 or 60 min after VSV8 peptide injection. Western blot analysis was performed on cytosolic and nuclear lysates for p50, p65 and RelB NF-κB proteins. The same amount of protein was added to each lane.

FIG. 6B is further analysis of the interaction of GST-IκBNS or GST-IκBα with NF-κB components in vivo and in vitro. Cytosolic and nuclear fractions of thymic lysates were incubated with GST (Ctl), GST-IκBNS or GST-IκBα proteins bound to beads and the interacting proteins identified by Western blot analysis with the indicated subunit-specific antibodies following SDS-PAGE.

FIG. 6C is an analysis of the interaction of IκBNS and IκBα with in vitro translated p50 and p65 proteins. [³⁵S]Radiolabeled murine NF-κB proteins were produced by in vitro translation and incubated individually or in combination with 5 μg GST, GST-IκBNS or GST-IκBα coupled to beads. Associated proteins were identified by autoradiography after SDS-PAGE. In each pair of lanes incubated with an NF-κB protein(s), the left lane shows binding to the GST control beads and the right lane shows interaction with GST-IκBNS-(upper panel) or GST-IκBα-(lower panel) beads.

FIG. 7A demonstrates that IκBNS antisense oligonucleotides block VSV8-induced negative selection in N15tg RAG-2^{-/-} H-2^{b} FTOC. FACS analysis of DP thymocytes in FTOC from Ni 5tg RAG-2^{-/-} H-2^{b} mice after no addition (None), VSV8 peptide addition for 4 h (VSV8), or VSV8 addition for 4 h following 18 h of antisense (AS+VSV8) or sense (S+VSV8) oligonucleotide treatment. The expression of CD4 (Y axis) or CD8 (X axis) was analyzed by two-color flow cytometry after gating on live cells. The percentage of DP thymocytes is indicated.

FIG. 7B represents the number of cells for each thymic lobe (Total), the DP population (DP) and the CD8 SP population (CD8 SP). One representative experiment of five is shown.

FIG. 8 is a model of the role of IκBNS in negative selection. Green arrows indicate TCR pathways in DP thymocytes induced by positively selecting stimuli while red arrows denote those of negatively selecting stimuli.

FIGS. 9A-9I are the human genomic sequence of IκBNS (SEQ ID NO: 1). The exons are indicated by shading. Exon I spans 1024-1215 bp; exon II spans 1297-1400 bp; exon III spans 1589-1734 bp; exon IV spans 1858-1911 bp; exon V spans 2001-2147 bp; exon VI spans 7637-7753 bp; exon VII spans 8073-8237 bp; and exon VIII spans 9515-9568 bp.

FIG. 10 is the mouse IκBNS cDNA sequence (SEQ ID NO: 3). The open reading frame (ORF) spans 473-1456 bp, and is indicated by shading.

FIG. 11 is the sequence alignment of human (SEQ ID NO: 2) and mouse (SEQ ID NO: 4) IκBNS protein sequences. In the center line between human and mouse sequences, amino acid identity is denoted by the identical amino acid; conservative amino acids are denoted by “+”; stretched gaps in the amino acid sequence are denoted by “−”; non-identity between amino acid sequences are denoted as blank spaces.

The foregoing and other objects, features and advantages of the invention will be apparent from the following more particular description of preferred embodiments of the invention.

### DETAILED DESCRIPTION OF THE INVENTION

Apoptosis is a morphologically stereotyped form of programmed cell death utilized by metazoan organisms during normal development as well as for homeostasis (Kerr et al., 1972; Horvitz et al., 1982; Steller, 1995; Vaux and Korsmeyer, 1999). Excess cells at each stage of organogenesis are eliminated by this mechanism. Within the hematopoetic system this phenomenon is well described (Cohen et al., 1992).

In T lymphoid development, αβ T cell receptors (TCR) are generated by a stochastic process of rearrangement of variable gene segments. Through combinatorial diversity, billions of different TCRs are generated ensuring protection against a myriad of infectious agents without a requirement to encode all TCR specificities in the germline (Davis and Bjorkman, 1988). One potential hazard of this diversity-generating mechanism is the creation of self-reactive TCRs. Thus, T cell repertoire generation must be carefully regulated through a developmental selection program termed negative selection (von Boehmer, 1991). Those TCRs that recognize self-MHC molecules and their associated peptides (pMHC) too strongly are eliminated by negative selection whereas those thymocytes with low affinity for self-MHC undergo maturation and are exported to the periphery in a process termed positive selection (Goldrath and Bevan, 1999). These selection processes act predominantly at the level of immature CD4+ CD8+ double positive (DP) thymocytes and appear to be biochemically distinct (Sebzda et al., 1999). TCRs and peptide-binding MHC molecules have co-evolved to interact with one another such that a given TCR can distinguish subtle differences between self and foreign (i.e., infectious) peptides bound to an MHC molecule (Reinherz et al., 1999). The TCR-related selection processes are complex, being dependent not only on the monomeric affinity of the TCR-pMHC interaction (Alam et al., 1996) but the interplay between co-stimulation, co-receptor ligation and TCR/pMHC affinity (Sebzda et al., 1999).

There are two major pathways of cell execution after the receipt of a death signal: the caspase enzymatic cascade and mitochondrial dysfunction (Gross et al., 1999). Bcl2 family members play a pivotal role in deciding whether a cell will live or die through their activities executed at the level of mitochondrial membrane permeability. However, over-expression of Bcl-2 and Bcl-xL in transgenic mice does not affect TCR-induced cell death suggesting that TCR-triggered death depends on caspase activity (Rathmell and Thompson, 1999). In fact, TCR triggering by “self-reactive” pMHC ligands during thymic negative selection activates a caspase in DP thymocytes (Clayton et al., 1997). Inhibition of this enzymatic activity with caspase 3-like inhibitors prevents antigen-induced death of DP thymocytes in fetal thymic organ culture (FTOC) from TCR transgenic mice as well as apoptosis induced by anti-TCR mAb crosslinking and corticosteroids in FTOC of normal C57BL/6 mice (Clayton et al., 1997). Hence a caspase mediates immature thymocyte susceptibility to cell death. Consistent with this data, gene deletion studies have shown that caspases 3, 8 and 9 are required for cell death during mammalian development (Vaux and Korsmeyer, 1999).

In mammalian cells, certain apoptotic pathways require gene transcription whereas others do not. Examples of the former are dexamethasone-induced or superantigen-induced lymphocyte death while apoptosis triggered by CD95 is an example of the latter (D'Adamio et al., 1992; Vaux and Korsmeyer, 1999). Since TCR triggering leads to the induction of gene expression, transcription factors would also be expected to play a role in thymocyte development and selection. The transcription factor IRF-1 appears to be involved in both positive and negative selection (Penninger et al., 1997) while nur77, a second transcription factor, is involved in negative selection processes (Liu et al., 1994; Amsen et al., 1999). Deletion of the nur77 gene in vivo (Lee et al., 1995) did not result in impaired thymic selection implying that other pathways may be operative in negative selection. In this regard, NF-κB transcription factors have been implicated in providing both survival and death signals (Ghosh et al., 1998) and are expressed at high levels in the thymus in a constitutively active state (Ivanov and Ceredig, 1992).

Negative selection eliminates thymocytes bearing autoreactive T cell receptors (TCR) via an apoptotic mechanism. To identify additional key genes expressed in thymocytes undergoing peptide-triggered selection, a TCR transgenic mouse system was utilized. Antigen-induced negative selection was induced (Ghendler et al., 1998) and subtractive cDNA cloning performed using representational difference analysis (RDA) (Lisitsyn et al., 1993; Hubank and Schatz, 1994). A subtracted library was prepared and a cDNA which encodes a novel functional inhibitor of NF-κB, termed IκBNS, was cloned. IκBNS, is rapidly expressed upon TCR-triggered but not dexamethasone- or y-irradiation-stimulated thymocyte death. The predicted protein contains 7 ankyrin repeats and is homologous to known IκB family members. In class I and class II MHC-restricted TCR transgenic mice, transcription of IκBNS is stimulated by peptides that trigger negative selection but not by those inducing positive selection (i.e., survival) or nonselecting peptides. IκBNS blocks transcription from NF-κB reporters, alters NF-κB electrophoretic mobility shifts and interacts with NF-κB proteins in thymic nuclear lysates following TCR stimulation. Inhibition of IκBNS translation blocks antigen-induced negative selection in fetal thymic organ culture.

NF-κB transcription factors are a highly conserved family of dimeric proteins found in many cell types. These inducible transcription factors activate various genes in response to proinflammatory and noxious stimuli (Ghosh et al., 1998; Baeuerle, 1998; Karin and Ben-Neriah, 2000). The remarkable ability of NF-κB proteins to respond quickly to cell surface perturbations is a function of the special regulation of NF-κB transcriptional activity by inhibitors called IκB proteins. NF-κB exists in the cytoplasm complexed with an IκB protein; upon the cell receiving a signal to activate NF-κB, IκB is phosphorylated and degraded via the proteasome. The free NF-κB then enters the nucleus and activates gene transcription by binding particular DNA sites (Ghosh et al., 1998; Karin and Ben-Neriah, 2000). Seven IκB proteins termed IκBα, IκBβ, IκBε, IκBγ, Bcl-3, MAIL (IκBζ) (Kitamura et al., 2000; Yamazaki et al., 2001) and Interleukin-1-inducible nuclear ankyrin-repeat protein (INAP) (Haruta et al., 2001) have been identified in mammalian cells (Ghosh et al., 1998). All seven proteins contain ankyrin domains which mediate protein-protein interaction with NF-κB homo- and heterodimers. IκBα is the best characterized member of this family and functions by blocking the nuclear localization signal and DNA binding of NF-κB proteins thus preventing nuclear entry and NF-κB-induced gene transcription until appropriate cell stimulation (Ghosh et al., 1998; Karin and Ben-Neriah, 2000). The IκB family members may function by different mechanisms. For example, IκBα, IκBβ and IκBκε bind strongly to p65- or c-Rel-containing dimers and Bcl-3 more strongly interacts with p52 and p50 homodimers (Ghosh et al., 1998). Whereas IκBα maintains the balance of cytoplasmic vs nuclear NF-κB by a dynamic shuttling process, IκBβ and IκBε act to sequester NF-κB proteins in the cytoplasm (Johnson et al., 1999; Huang et al., 2000; Tam and Sen, 2001). Bcl-3 instead is a nuclear protein and acts to increase transcription of genes with NF-κB-sensitive promoters perhaps by removing the inhibitory p50 or p52 homodimers from DNA, allowing active p65/p50 heterodimers access to NF-κB sites. Furthermore, Bcl-3 contains transactivation domains and may form activating complexes with p50 and p52 (Bours et al., 1993; Fujita et al., 1993). Other nuclear co-regulators bind Bcl-3 suggesting that Bcl-3 may act as a bridging factor between other proteins and p50 or p52 (Dechend et al., 1999). IκBγ has a limited distribution and is as yet not well characterized (Ghosh et al., 1998).

The role of the NF-κB/IκB transcriptional regulatory pathways in T cell development is currently unclear. Gene knockouts of individual NF-κB family members do not demonstrate marked defects in lymphocyte development, but this lack of a thymic phenotype may result from functional redundancy (Ghosh et al., 1998) since double knockouts demonstrate more severe phenotypes (Gerondakis et al., 1999). For example, in p50/RelB and p50/p52 double knockouts, thymic atrophy was observed (Franzoso et al., 1997; Weih et al., 1997). In transgenic mice expressing a dominant negative IκBα in the thymus, thymocytes developed normally although partial defects in proliferation were observed (Hettmann et al., 1999) particularly in CD44-CD25-DN thymocytes (Voll et al., 2000). Analysis of IKKβ negative fetal liver stem cells in reconstitution experiments suggests a similar proliferation defect in thymocytes with altered NF-κB activity (Senftleben et al., 2001). IKKβ is a catalytic subunit of the IκB kinase complex that phosphorylates IκB proteins in the first step of the proteasome degradation pathway. Thus IKKβ negative cells should be functionally equivalent to cells containing super-repressor IκBα. The current consensus seems to be that NF-κB proteins play a role in lymphocyte development and affect survival of developing thymocytes through effects on proliferation, protection from TNF-induced apoptosis and regulation of anti-apoptotic genes such as bcl-xL (Hettmann et al., 1999; Senftleben et al., 2001; Voll et al., 2000).

The distribution of IκBNS expression suggests a more restricted role for this protein in NF-κB regulation during T cell development. Furthermore, the correlation of IκBNS induction with TCR-induced negative selection signals and the ability of IκBNS antisense oligonucleotides to block negative selection implies a very specific function in determining death/survival of immature thymocytes. Previous analyses of NF-κB inhibition by expression of a super-repressor IκBα which lacks IKK phosphorylation sites have suggested that NF-κB inhibition affects the proliferation of thymocytes during development but does not disrupt development itself (Hettmann et al., 1999; Bakker et al., 1999; Ferreira et al., 1999; Voll et al., 2000). An effect on apoptosis was also noted but while adenovirus-mediated expression of super-repressor IκBα in FTOC resulted in increased apoptosis (Bakker et al., 1999), a superrepressor IκBα transgene expressed under the control of a CD2 promoter resulted in a decrease in anti-CD3-mediated thymocyte death (Hettmann et al., 1999). Nonetheless, the thymocyte populations affected by NF-κB inhibition appear to be those subpopulations expressing the highest levels of NF-κB activity (Voll et al., 2000).

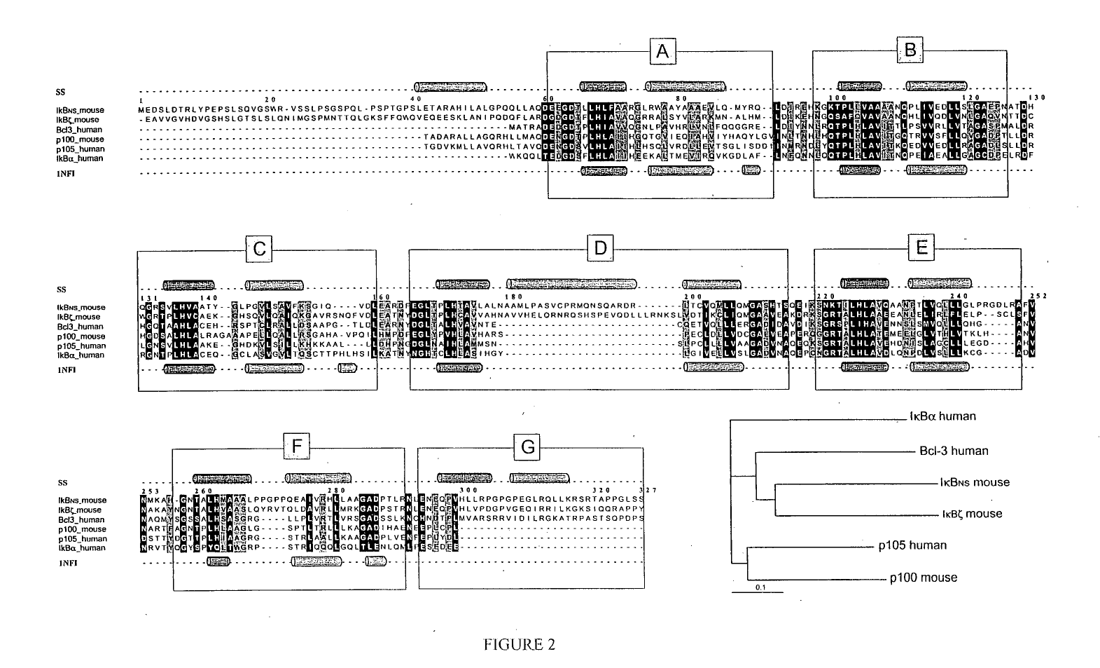

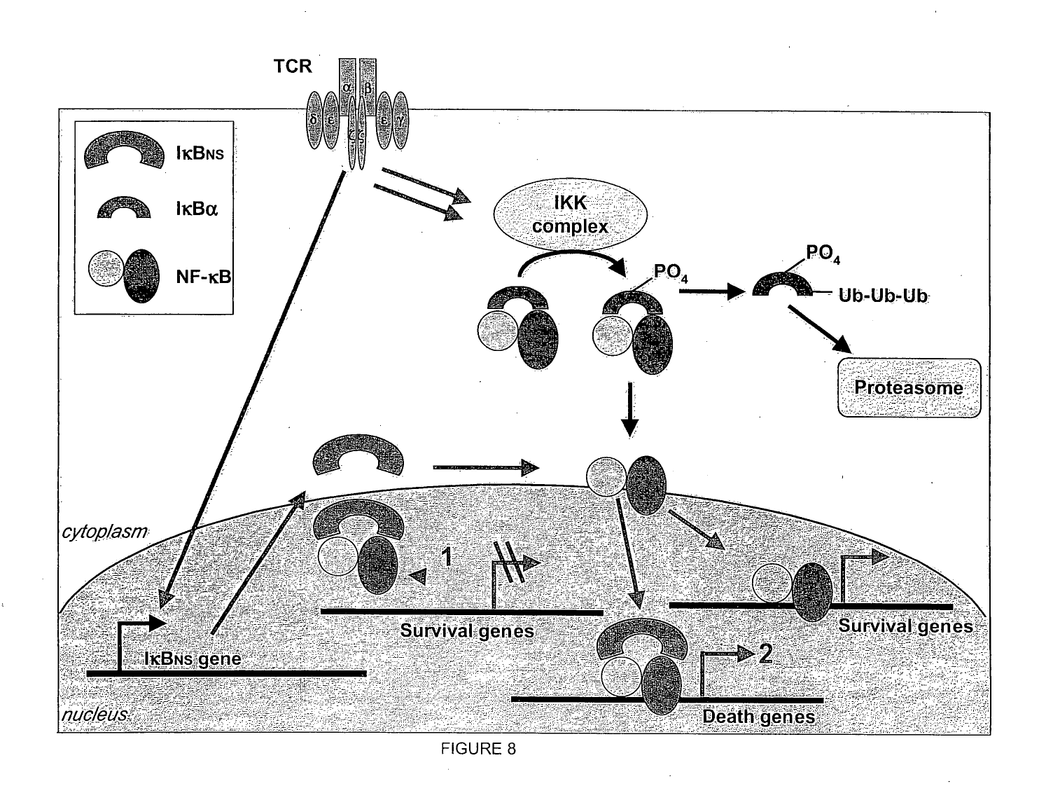

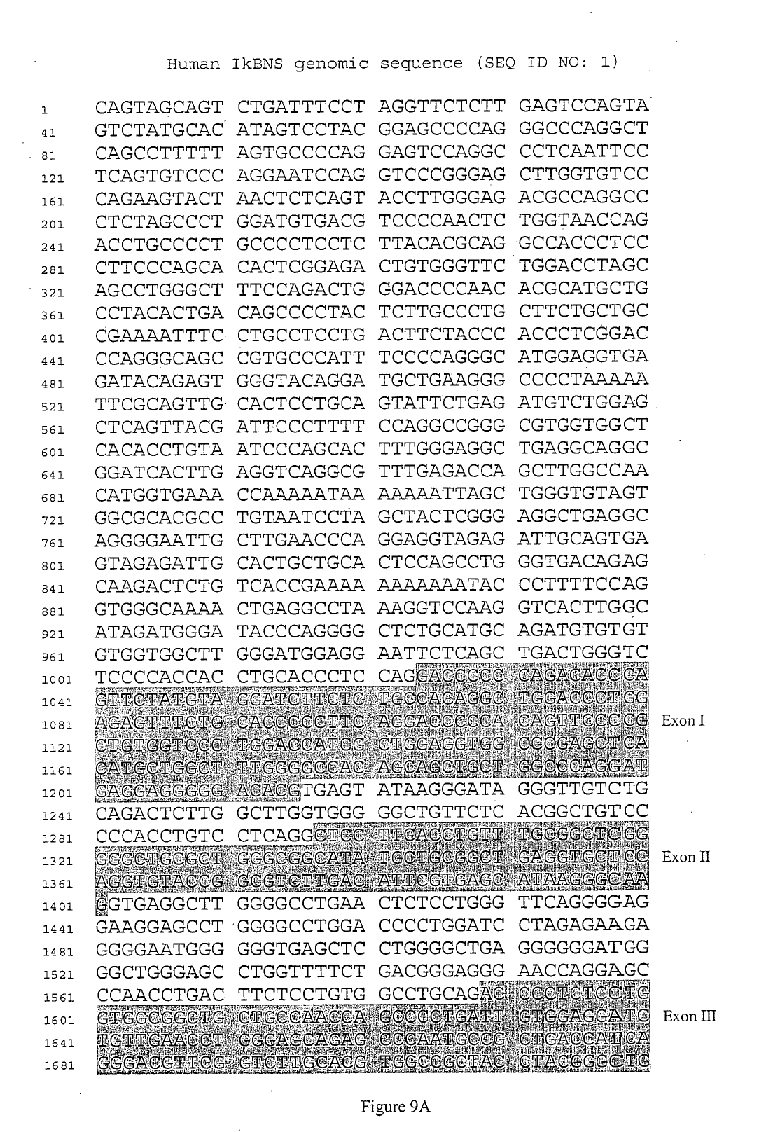

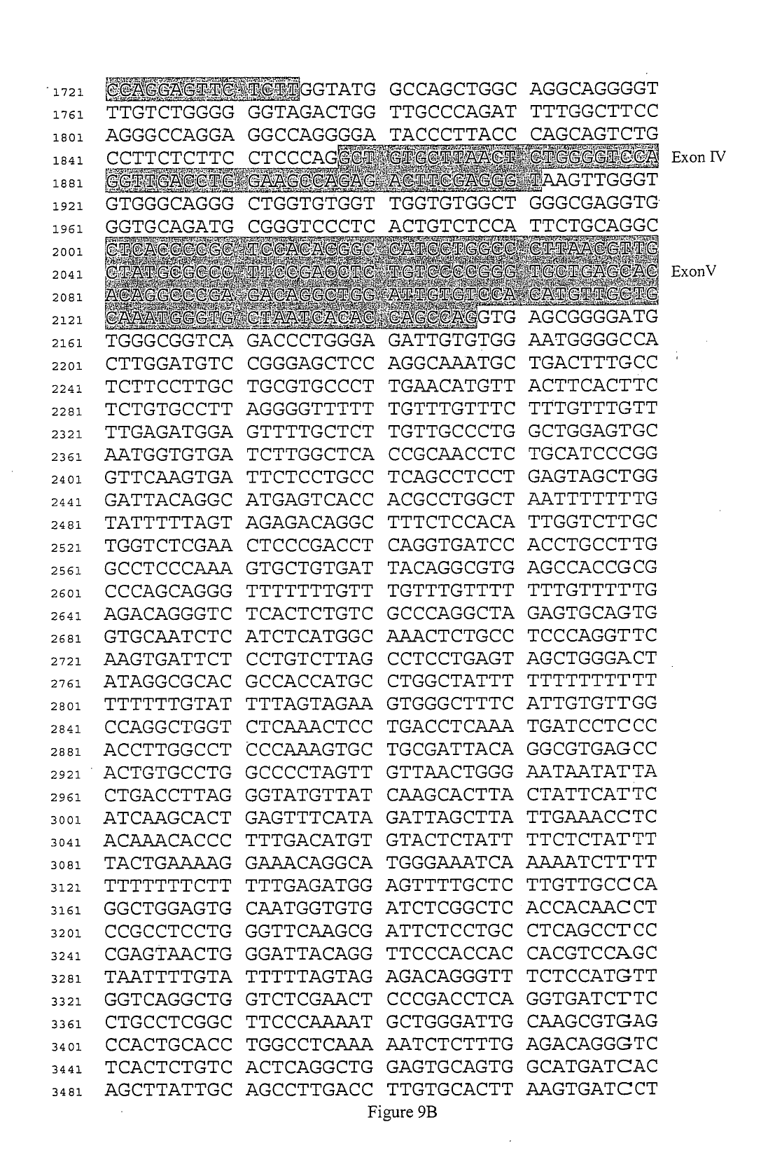

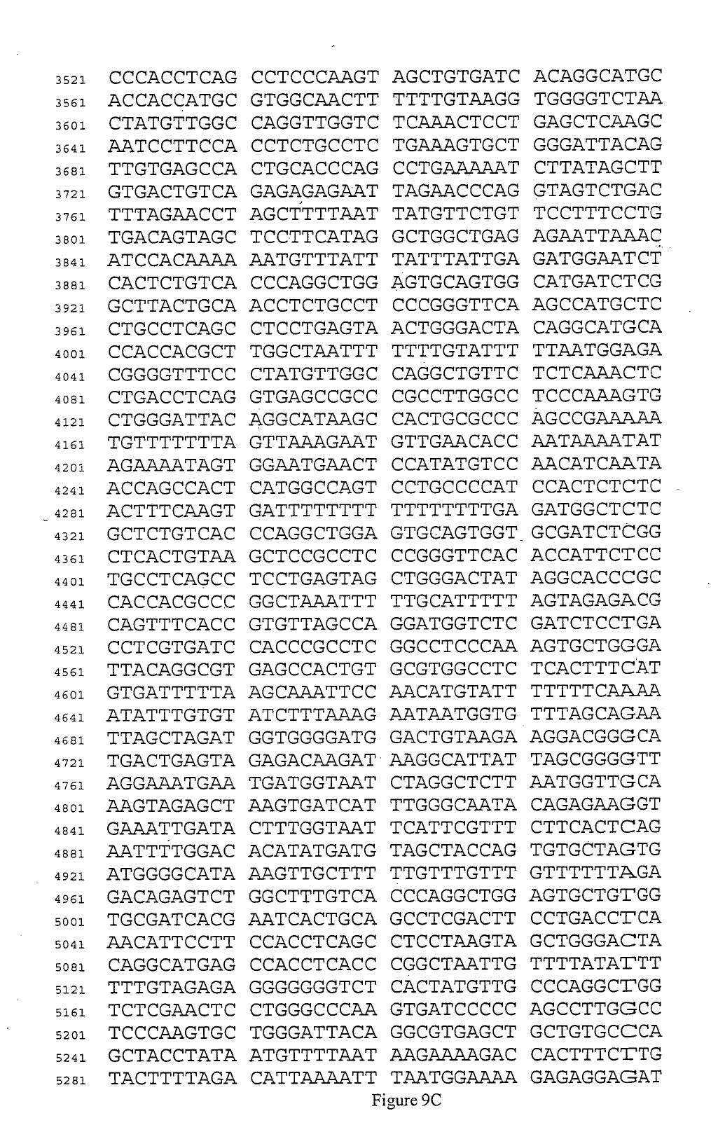

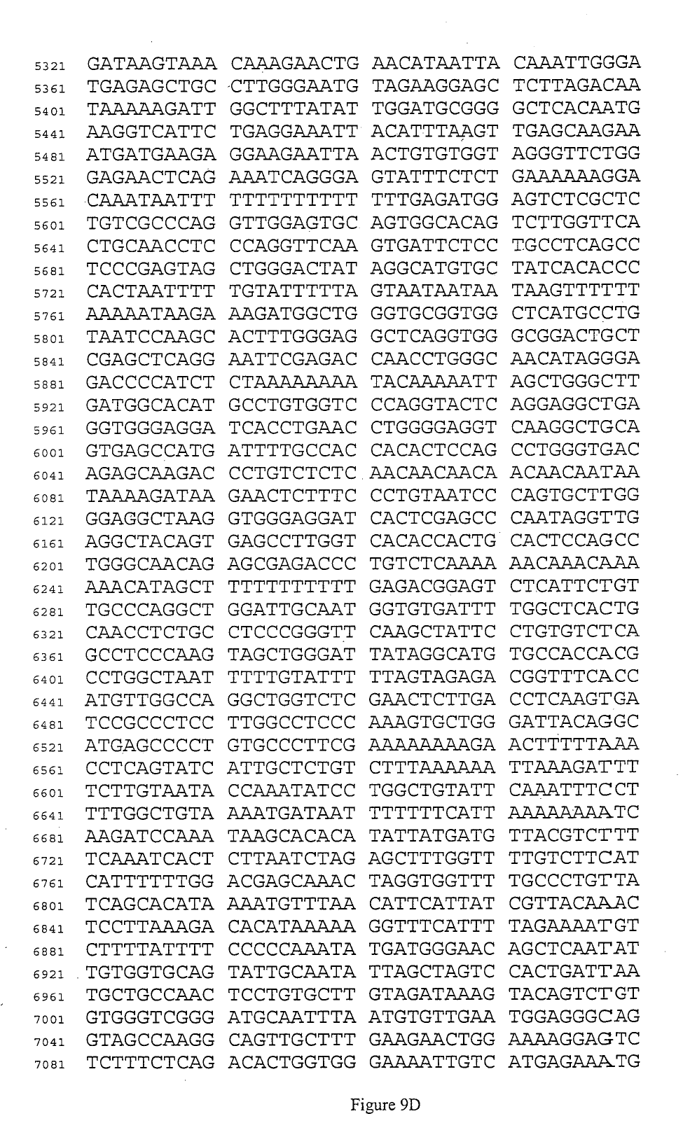

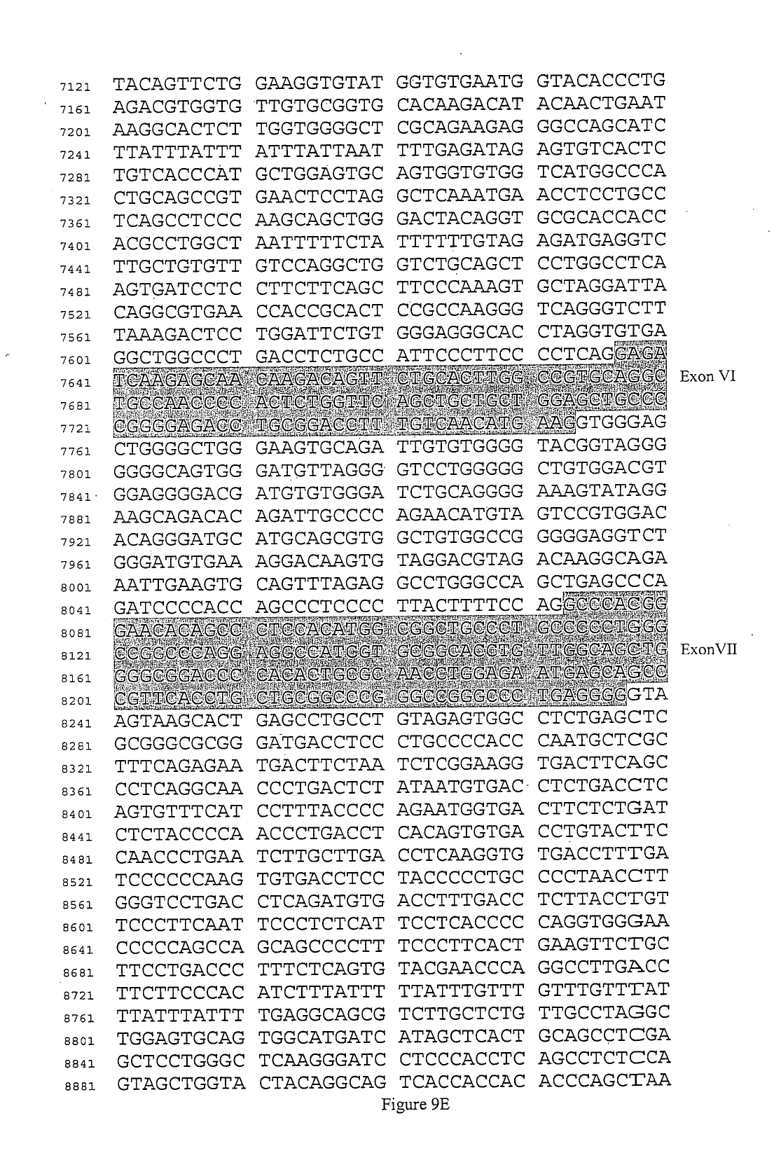

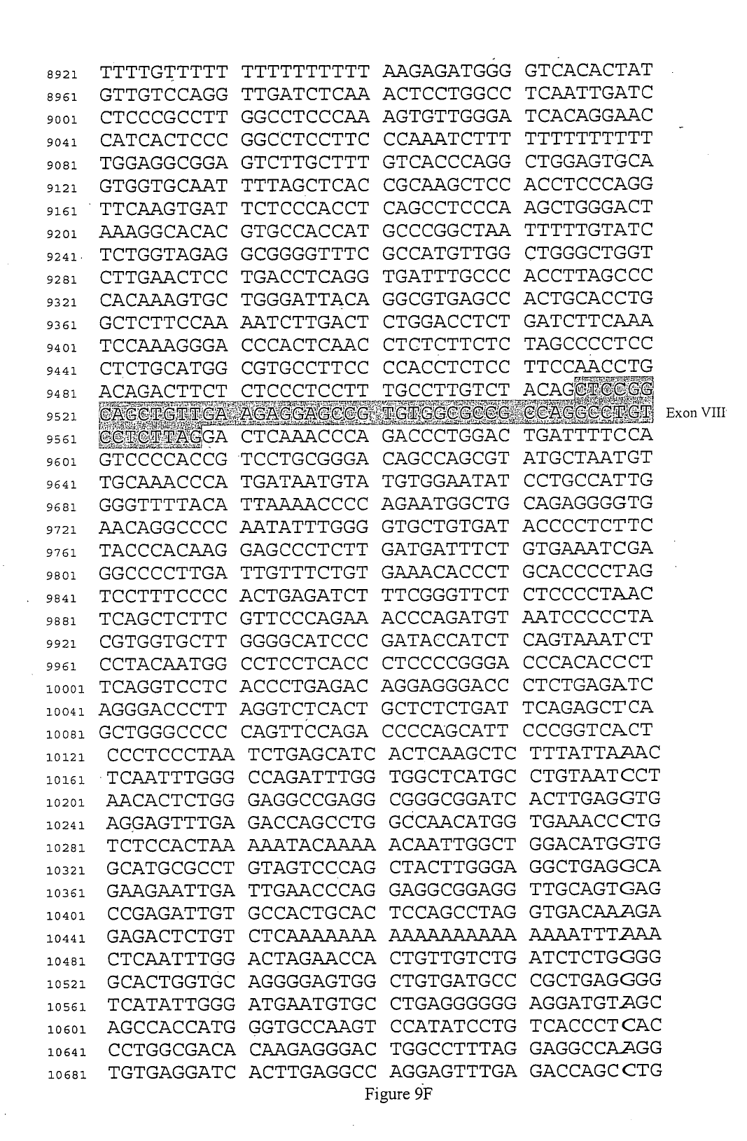

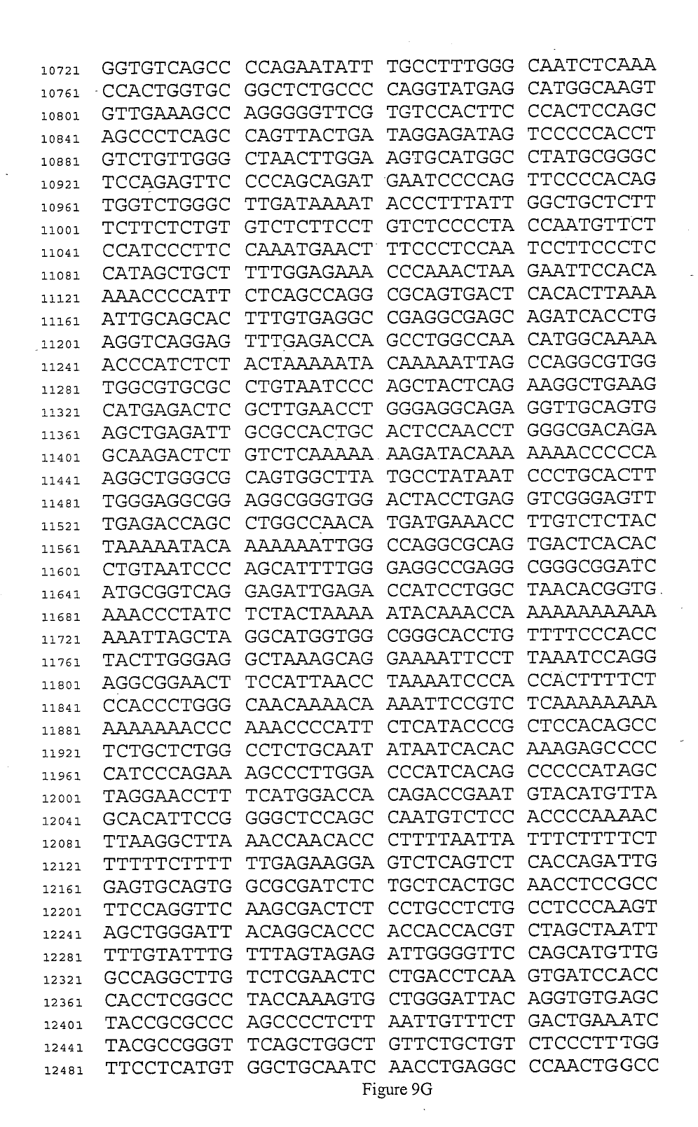

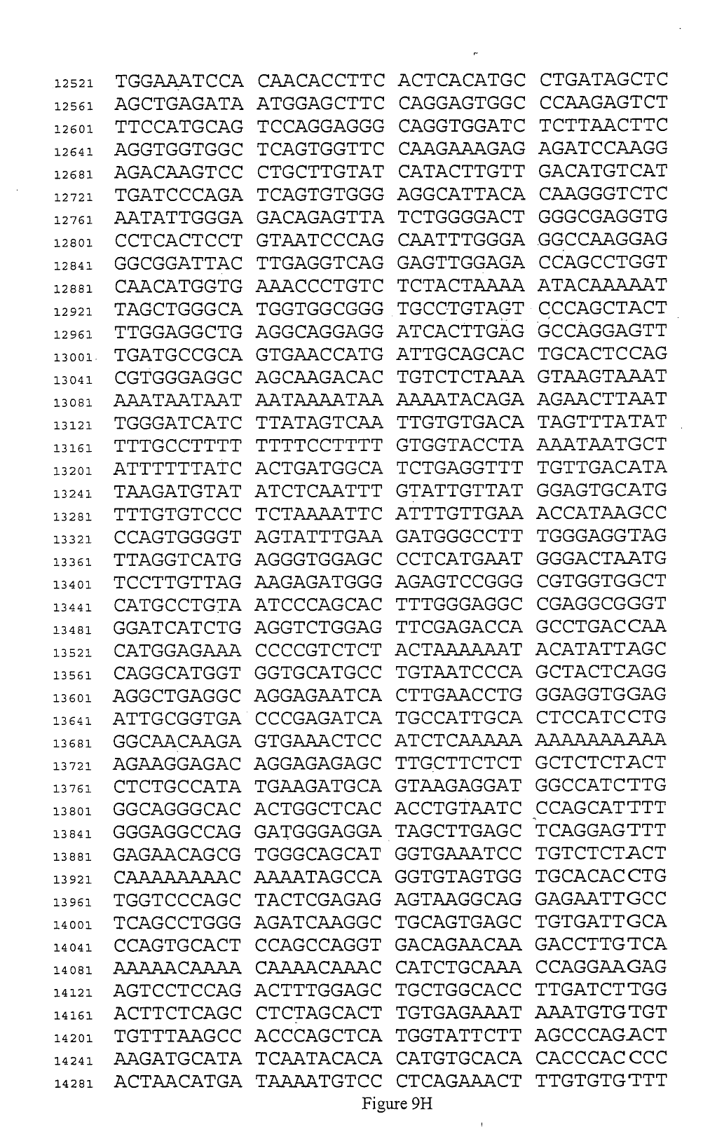

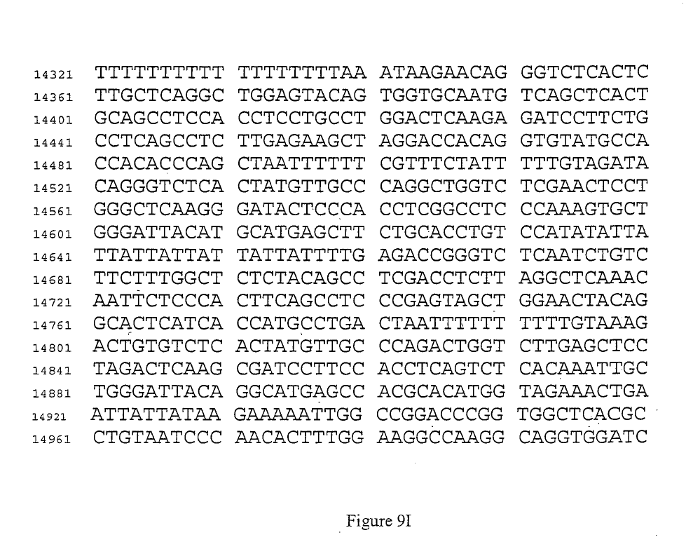

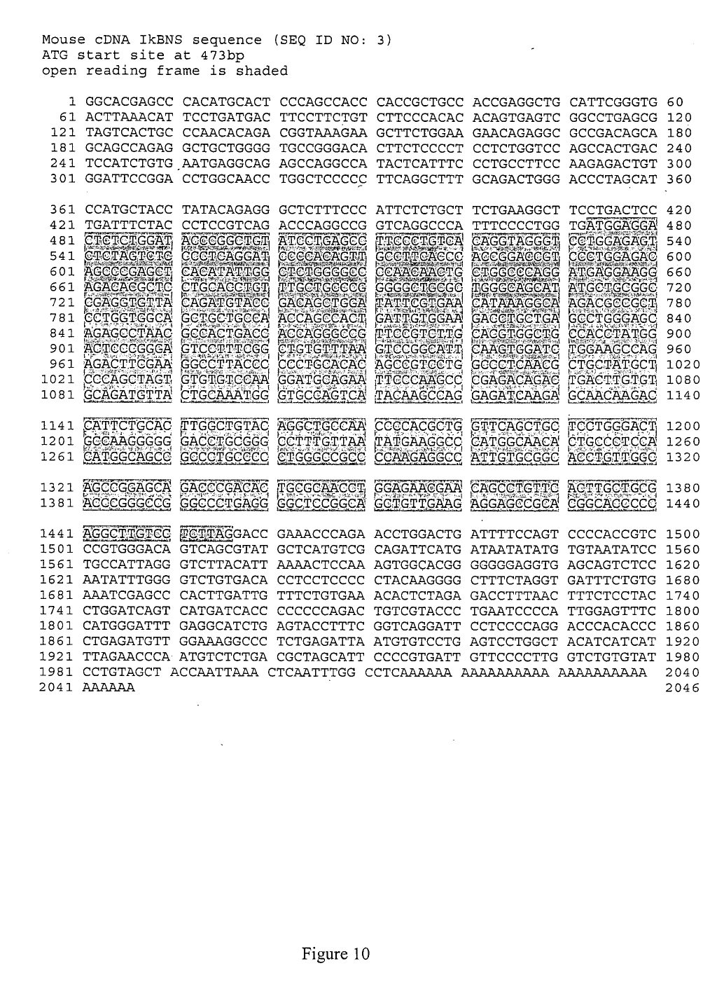

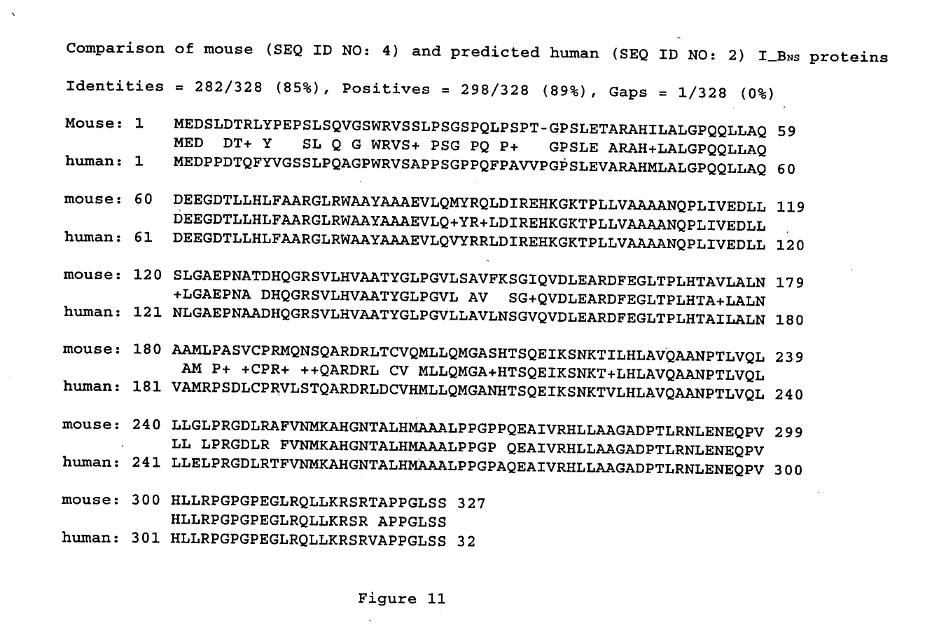

If NF-κB provides survival signals to developing thymocytes, then induction of an inhibitor of this activity would clearly result in cell death. However, the IκBα superrepressor transgene prevented anti-CD3ε-triggered cell death (Hettmann et al., 1999). Thus simply inhibiting NF-κB activation does not appear to result in TCR-triggered thymocyte apoptosis, at least as stimulated by anti-CD3ε mAb crosslinking. To the contrary, these findings suggest that NF-κB activation is required for TCR-triggered thymocyte apoptosis. One explanation for this paradox is that IκBNS may behave in a different manner than IκBα. IκBNS, like Bcl-3 with which it is most structurally homologous (FIG. 2), may bind to p50 and p52 homodimers. As described above, these NF-κB complexes act as inhibitors of transcription by blocking NF-κB sites on DNA. IκBNS may remove these inactive complexes from DNA and redirect TCR-triggered gene transcription (Franzoso et al., 1992; Bours et al., 1993; Fujita et al., 1993). Alternatively, IκBNS may also act as a bridging component between NF-κB dimers and other proteins. One model for the role of IκBNS in negative selection is offered in FIG. 8. Upon receipt of a positively selecting signal through the TCR, IKK is activated. IκBα is then degraded allowing NF-κB to translocate to the nucleus and induce transcription of genes required for survival (as indicated by the green arrows). However, upon receipt of a negatively selecting signal, transcription of IκBNS is induced in addition (as indicated by the red arrows). We believe that IκBNS has two potential modes of action. IκBNS enters the nucleus, binds to NF-κB/DNA complexes and removes these active complexes from the DNA thereby blocking transcription of the “survival genes” (mechanism 1). A second possibility is that IκBNS binds NF-κB dimers and redirects the active complex to a new site resulting in the transcription of genes which execute the cell (mechanism 2). IκBNS may induce these “execution” genes on its own or require an additional factor(s) present in DP thymocytes.

With respect to the panel of peptides tested herein, a similar pattern of mRNA induction for IκBNS and nur77 was observed, a transcription factor also linked to negative selection processes (Liu et al., 1994; Amsen et al., 1999). The similar pattern of gene expression suggests that the same TCR signal may induce nur77 and IκBNS as a consequence of activation of a very early upstream gene which regulates both or that TCR-triggered post-translational modification of a protein(s) then activates these two genes directly. From characterization of the RDA products generated herein, nur77 and IκBNS account for the vast majority of differentially expressed clones. For the current analysis N15tg RAG-2^{-/-} TCR H-2^{b} transgenic mice bearing a single TCR recognizing the VSV8 peptide complexed with the class I MHC K^{b} molecule were utilized. A strength of this system is the accessibility of differentially selecting peptide ligands and the detailed knowledge available concerning the affects of small structural alterations of peptide sidechains on the signal transmitted to the T cell or thymocyte (Ghendler et al., 1998; Sasada et al., 2000). This information allows the correlation of the induction of IκBNS with those peptide stimuli which trigger negative selection in N15tg RAG-2^{-/-} TCR H-2^{b} transgenic mice. Similarly, in the class II system, TCRCyt5CC7-I-RAG-2^{-/- H-}2^{a} mice injected with the negatively selecting PCC peptide also express IκBNS message in the thymus within 1 h of treatment. Thus IκBNS serves as a molecular marker of negative selection in both class I and class II TCR transgenic mice. In wild type C57BL/6 mice, IκBNS is induced by anti-CD3ε injection, indicating a role for IκBNS in negative selection of non-transgenic mice as well. That IκBNS gene expression is strongly linked to TCR-triggered negative selection and not other forms of apoptosis is clear from the observation that γ-irradiation and dexamethasone fail to induce IκBNS message in thymocytes. Transcription of two recently identified IκB-like genes is also induced by cell surface triggering; MAIL (IκBζ) is induced by LPS (Kitamura et al., 2000; Yamazaki et al., 2001) and INAP is induced by IL-1 (Haruta et al., 2001).

That TCR triggering differentially induces transcription of genes and biochemical signaling events predicated on the extent of TCR-crosslinking or ligand affinity is well documented (Meuer et al., 1984; Fujita et al., 1986; Sloan-Lancaster et al., 1994; Gong et al., 2001). Correlative studies suggest that the TCR signal required to induce positive selection necessitates a weaker TCR-pMHC interaction than that required for negative selection (Alam et al., 1996). Thus, exceeding a particular affinity threshold leads to negative selection. In this respect, IκBNS appears to be a qualitative transducer of divergent TCR ligation signals: those signals resulting in negative selection induce transcription of this gene while nonselecting or positively selecting peptides for which the TCR has less affinity do not.

As described herein, novel nucleic acid molecules have been identified from thymocytes triggered to undergo negative selection. These nucleic acid molecules, or a subset thereof, can serve as markers to identify and characterize negative selection of T cells.

In one embodiment, the nucleic acid molecules consist of a nucleotide sequence selected from the group consisting of SEQ ID NOS: 1 and 3, the complement of SEQ ID NOS: 1 and 3, nucleic acid molecules that encode SEQ ID NOS: 2 and 4, and a nucleic acid molecule consisting of exons I to XIII of SEQ ID NO: 1 in a contiguous sequence, or combinations or permutations of exons I to XIII of SEQ ID NO: 1 in a contiguous sequence. In another embodiment, the nucleic acid molecules comprise a nucleotide sequence selected from the group consisting of SEQ ID NO: 3, the complement of SEQ ID NO: 3, and nucleic acid molecules that encode SEQ ID NOS: 2 and 4, and a nucleic acid molecule consisting of exons I to XIII of SEQ ID NO: 1 in a contiguous sequence, or combinations or permutations of exons I to XIII of SEQ ID NO: 1 in a contiguous sequence. In another embodiment, the nucleic acid molecule hybridizes under high stringency conditions to a nucleotide sequence selected from the group consisting of SEQ ID NOS: 1 and 3, the complement of SEQ ID NOS: 1 and 3, and nucleic acid molecules that encode SEQ ID NOS: 2 and 4. In a further embodiment, the nucleic acid molecule of the invention comprises a nucleotide sequence which is greater than about 75 percent, and more preferably greater than about 80 percent, and even more preferably greater than about 90 percent, identical to a nucleotide sequence selected from the group consisting of SEQ ID NOS: 1 and 3, the complement of SEQ ID NOS: 1 and 3, and nucleic acid molecules that encode SEQ ID NOS: 2 and 4. In a preferred embodiment, the nucleic acid molecule which hybridizes under conditions of high stringency to a nucleotide sequence selected from the group consisting of SEQ ID NOS: 1 and 3, the complement of SEQ ID NOS: 1 and 3, and the nucleic acid molecules that encode SEQ ID NOS: 2 and 4 is isolated from mammalian tissue.

The invention further relates to an isolated portion of any of SEQ ID NOS: 1 and 3, the complement of SEQ ID NOS: 1 and 3, and nucleic acid molecules that encode SEQ ID NOS: 2 and 4, which portion is sufficient in length to distinctly characterize the sequence. For example, the isolated portion can be from about 15 to about 25 nucleotides in length, nucleotides in length, and more preferably from about 25 to about 40 nucleotides in length.

As appropriate, nucleic acid molecules of the present invention can be RNA, for example, mRNA, or DNA, such as cDNA and genomic DNA. DNA molecules can be double-stranded or single-stranded; single stranded RNA or DNA can be the coding or sense strand, or the non-coding or antisense, strand. Preferably, the nucleic acid molecule comprises at least about 10 nucleotides, more preferably at least about 50 nucleotides, and even more preferably at least about 200 nucleotides. The nucleic acid molecule can include all or a portion of the coding sequence of a gene and can further comprise additional non-coding sequences such as introns and non-coding 3′ and 5′ sequences (including regulatory sequences, for example). Additionally, the nucleic acid molecule can be fused to a marker sequence, for example, a sequence which encodes a polypeptide to assist in isolation or purification of the polypeptide. Such sequences include, but are not limited to, those which encode a glutathione-S-transferase (GST) fusion protein and hemophilus influenza hemaglutinin tag (HA).

As used herein, an “isolated” gene or nucleic acid molecule is intended to mean a gene or nucleic acid molecule which is not flanked by nucleic acid molecules which normally (in nature) flank the gene or nucleic acid molecule (such as in genomic sequences) and/or has been completely or partially purified from other transcribed sequences (as in a cDNA or RNA library). For example, an isolated nucleic acid of the invention may be substantially isolated with respect to the complex cellular milieu in which it naturally occurs. In some instances, the isolated material will form part of a composition (for example, a crude extract containing other substances), buffer system or reagent mix. In other circumstance, the material may be purified to essential homogeneity, for example as determined by PAGE or column chromatography such as HPLC. Preferably, an isolated nucleic acid comprises at least about 50, 80 or 90 percent (on a molar basis) of all macromolecular species present. Thus, an isolated gene or nucleic acid molecule can include a gene or nucleic acid molecule which is synthesized chemically or by recombinant means. Thus, recombinant DNA contained in a vector are included in the definition of “isolated” as used herein. Also, isolated nucleic acid molecules include recombinant DNA molecules in heterologous host cells, as well as partially or substantially purified DNA molecules in solution. In vivo and in vitro RNA transcripts of the DNA molecules of the present invention are also encompassed by “isolated” nucleic acid molecules. Such isolated nucleic acid molecules are useful in the manufacture of the encoded protein, as probes for isolating homologous sequences (e.g., from other mammalian species), for gene mapping (e.g., by in situ hybridization with chromosomes), or for detecting expression of the gene in tissue (e.g., human tissue such as brain tissue), such as by Northern blot analysis.

The invention described herein also relates to fragments or portions of the isolated nucleic acid molecules described above. The term “fragment” is intended to encompass a portion of a nucleic acid molecule described herein which is from at least about 25 contiguous nucleotides to at least about 40 contiguous nucleotides or longer in length; such fragments are useful as probes, e.g., for diagnostic methods and also as primers. Particularly preferred primers and probes selectively hybridize to nucleic acid molecules comprising the nucleotide sequences of any of SEQ ID NOS: 1 and 3, the complement of SEQ ID NOS: 1 and 3, and nucleic acid molecules that encode SEQ ID NOS: 2 and 4. For example, fragments which encode antigenic proteins or polypeptides described herein are useful.

The invention also pertains to nucleic acid molecules which hybridize under high stringency hybridization conditions (e.g., for selective hybridization) to a nucleotide sequence described herein. Hybridization probes are oligonucleotides which bind in a base-specific manner to a complementary strand of nucleic acid. Such probes include polyamide nucleic acids, as described in Nielsen et al., Science 254, 1497-1500 (1991). Appropriate stringency conditions are known to those skilled in the art or can be found in standard texts such as Current Protocols in Molecular Biology, John Wiley & Sons, N.Y. (1989), 6.3.1-6.3.6. For example, stringent hybridization conditions include a salt concentration of no more than 1 M and a temperature of at least 25° C. In one embodiment, conditions of 5×SSPE (750 mM NaCl, 50 mM NaPhosphate, 5 mM EDTA, pH 7.4) and a temperature of 25-30° C., or equivalent conditions, are suitable for specific probe hybridizations. Equivalent conditions can be determined by varying one or more of the parameters given as an example, as known in the art, while maintaining a similar degree of identity or similarity between the target nucleic acid molecule and the primer or probe used. Hybridizable nucleic acid molecules are useful as probes and primers for diagnostic applications.

As used herein, the term “primer” refers to a single-stranded oligonucleotide which acts as a point of initiation of template-directed DNA synthesis under appropriate conditions (e.g., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

Accordingly, the invention pertains to nucleic acid molecules which have a substantial identity with the nucleic acid molecules described herein; particularly preferred are nucleic acid molecules which have at least about 90%, and more preferably at least about 95% identity with nucleic acid molecules described herein. Thus, DNA molecules which comprise a sequence which is different from the naturally-occurring nucleic acid molecule but which, due to the degeneracy of the genetic code, encode the same protein or polypeptide are the subject of this invention. The invention also encompasses variations of the nucleic acid molecules of the invention, such as those encoding portions, analogues or derivatives of the encoded protein or polypeptide. Such variations can be naturally-occurring, such as in the case of allelic variation, or non-naturally-occurring, such as those induced by various mutagens and mutagenic processes. Variations include, but are not limited to, addition, deletion and substitution of one or more nucleotides which can result in conservative or non-conservative amino acid changes, including additions and deletions. Preferably, the nucleotide or amino acid variations are silent; that is, they do not alter the characteristics or activity of the encoded protein or polypeptide. As used herein, activities of the encoded protein or polypeptide include, but are not limited to, catalytic activity, binding function, antigenic function and oligomerization function.

The nucleotide sequences of the nucleic acid molecules described herein, e.g., SEQ ID NOS: 1 and 3, the complement of SEQ ID NOS: 1 and 3, and nucleic acid molecules that encode SEQ ID NOS: 2 and 4, can be amplified by methods known in the art. For example, this can be accomplished by e.g., PCR. See generally PCR Technology. Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be radiolabeled and used as a probe for screening a cDNA library derived from thymus tissue, e.g., human thymus tissue, mRNA in λzap express, ZIPLOX or other suitable vector. Corresponding clones can be isolated, DNA can obtained following in vivo excision, and the cloned insert can be sequenced in either or both orientations by art recognized methods, to identify the correct reading frame encoding a protein of the appropriate molecular weight. For example, the direct analysis of the nucleotide sequence of nucleic acid molecules of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the protein(s) and the DNA encoding the protein can be isolated, sequenced and further characterized.

With respect to protein or polypeptide identification, bands identified by gel analysis can be isolated and purified by HPLC, and the resulting purified protein can be sequenced. Alternatively, the purified protein can be enzymatically digested by methods known in the art to produce polypeptide fragments which can be sequenced. The sequencing can be performed, for example, by the methods of Wilm et al. (Nature 379(6564):466-469 (1996)). The protein may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

In addition to substantially full-length polypeptides encoded by nucleic acid molecules described herein, the present invention includes biologically active fragments of the polypeptides, or analogs thereof, including organic molecules which simulate the interactions of the polypeptides. Biologically active fragments include any portion of the full-length polypeptide which confers a biological function on the variant gene product, including ligand binding, and antibody binding. Ligand binding includes binding by nucleic acids, proteins or polypeptides, small biologically active molecules, or large cellular structures.

This invention also pertains to an isolated protein or polypeptide encoded by the nucleic acid molecules of the invention. The encoded proteins or polypeptides of the invention can be partially or substantially purified (e.g., purified to homogeneity), and/or are substantially free of other proteins. According to the invention, the amino acid sequence of the polypeptide can be that of the naturally-occurring protein or can comprise alterations therein. Such alterations include conservative or non-conservative amino acid substitutions, additions and deletions of one or more amino acids; however, such alterations should preserve at least one activity of the encoded protein or polypeptide, i.e., the altered or mutant protein should be an active derivative of the naturally-occurring protein. For example, the mutation(s) can preferably preserve the three dimensional configuration of the binding and/or the ankyrin repeats of the native protein. The presence or absence of biological activity or activities can be determined by various functional assays as described herein. Moreover, amino acids which are essential for the function of the encoded protein or polypeptide can be identified by methods known in the art. Particularly useful methods include identification of conserved amino acids in the family or subfamily, site-directed mutagenesis and alanine-scanning mutagenesis (for example, Cunningham and Wells, Science 244:1081-1085 (1989)), crystallization and nuclear magnetic resonance. The altered polypeptides produced by these methods can be tested for particular biologic activities, including immunogenicity and antigenicity.

Specifically, appropriate amino acid alterations can be made on the basis of several criteria, including hydrophobicity, basic or acidic character, charge, polarity, size, the presence or absence of a functional group (e.g., —SH or a glycosylation site), and aromatic character. Assignment of various amino acids to similar groups based on the properties above will be readily apparent to the skilled artisan; further appropriate amino acid changes can also be found in Bowie et al. (Science 247:1306-1310(1990)).

The encoded polypeptide can also be a fusion protein comprising all or a portion of the amino acid sequence fused to an additional component. Additional components, such as radioisotopes and antigenic tags, can be selected to assist in the isolation or purification of the polypeptide or to extend the half life of the polypeptide; for example, a hexahistidine tag would permit ready purification by nickel chromatography. Furthermore, polypeptides of the present invention can be progenitors of the active protein; progenitors are molecules which are cleaved to form an active molecule.

Polypeptides described herein can be isolated from naturally-occurring sources, chemically synthesized or recombinantly produced. Polypeptides or proteins of the present invention can be used as a molecular weight marker on SDS-PAGE gels or on molecular sieve gel filtration columns using art-recognized methods.

The invention also provides expression vectors containing a nucleic acid sequence described herein, operably linked to at least one regulatory sequence. Many such vectors are commercially available, and other suitable vectors can be readily prepared by the skilled artisan. “Operably linked” is intended to meant that the nucleic acid molecule is linked to a regulatory sequence in a manner which allows expression of the nucleic acid sequence. Regulatory sequences are art-recognized and are selected to produce the encoded polypeptide or protein. Accordingly, the term “regulatory sequence” includes promoters, enhancers, and other expression control elements which are described in Goeddel, Gene Expression Technology: Methods in Enzymology 185, Academic Press, San Diego, Calif. (1990). For example, the native regulatory sequences or regulatory sequences native to the transformed host cell can be employed. It should be understood that the design of the expression vector may depend on such factors as the choice of the host cell to be transformed and/or the type of protein desired to be expressed. For instance, the polypeptides of the present invention can be produced by ligating the cloned gene, or a portion thereof, into a vector suitable for expression in either prokaryotic cells, eukaryotic cells or both (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17). Typically, expression constructs will contain one or more selectable markers, including, but not limited to, the gene that encodes dihydrofolate reductase and the genes that confer resistance to neomycin, tetracycline, ampicillin, chloramphenicol, kanamycin and streptomycin resistance.

Prokaryotic and eukaryotic host cells transfected by the described vectors are also provided by this invention. For instance, cells which can be transfected with the vectors of the present invention include, but are not limited to, bacterial cells such as E. coli (e.g., E. coli K12 strains), Streptomyces, Pseudomonas, Serratia marcescens and Salmonella typhimurium, insect cells (baculovirus), including Drosophila, fungal cells, such as yeast cells, plant cells and mammalian cells, such as thymocytes, Chinese hamster ovary cells (CHO), and COS cells.

Thus, a nucleic acid molecule described herein can be used to produce a recombinant form of the protein via microbial or eukaryotic cellular processes. Ligating the nucleic acid molecule into a gene construct, such as an expression vector, and transforming or transfecting into hosts, either eukaryotic (yeast, avian, insect, plant or mammalian) or prokaryotic (bacterial cells), are standard procedures used in producing other well known proteins. Similar procedures, or modifications thereof, can be employed to prepare recombinant proteins according to the present invention by microbial means or tissue-culture technology. Accordingly, the invention pertains to the production of encoded proteins or polypeptides by recombinant technology.

The proteins and polypeptides of the present invention can be isolated or purified (e.g., to homogeneity) from recombinant cell culture by a variety of processes. These include, but are not limited to, anion or cation exchange chromatography, ethanol precipitation, affinity chromatography and high performance liquid chromatography (HPLC). The particular method used will depend upon the properties of the polypeptide and the selection of the host cell; appropriate methods will be readily apparent to those skilled in the art.

The present invention also relates to antibodies which bind a polypeptide or protein of the invention. For instance, polyclonal and monoclonal antibodies, including non-human and human antibodies, humanized antibodies, chimeric antibodies and antigen-binding fragments thereof (Current Protocols in Immunology, John Wiley & Sons, N.Y. (1994); EP Application 173,494 (Morrison); International Patent Application WO86/01533 (Neuberger); and U.S. Pat. No. 5,225,539 (Winters)) which bind to the described protein or polypeptide are within the scope of the invention. A mammal, such as a mouse, rat, hamster, goat or rabbit, can be immunized with an immunogenic form of the protein (e.g., the full length protein or a polypeptide comprising an antigenic fragment of the protein which is capable of eliciting an antibody response). Techniques for conferring immunogenicity on a protein or polypeptide include conjugation to carriers or other techniques well known in the art. The protein or polypeptide can be administered in the presence of an adjuvant. The progress of immunization can be monitored by detection of antibody titers in plasma or serum. Standard ELISA or other immunoassays can be used with the immunogen as antigen to assess the levels of antibody.

Following immunization, anti-peptide antisera can be obtained, and if desired, polyclonal antibodies can be isolated from the serum. Monoclonal antibodies can also be produced by standard techniques which are well known in the art (Kohler and Milstein, Nature 256:495-497 (1975); Kozbar et al., Immunology Today 4:72 (1983); and Cole et al., Monoclonal Antibodies and Cancer Therapy, Alan R. Liss, Inc., pp. 77-96 (1985)). The term “antibody” as used herein is intended to include fragments thereof, such as Fab and F(ab)₂. Antibodies described herein can be used to inhibit the activity of the polypeptides and proteins described herein, particularly in vitro and in cell extracts, using methods known in the art. Additionally, such antibodies, in conjunction with a label, such as a radioactive label, can be used to assay for the presence of the expressed protein in a cell from, e.g., a tissue sample, and can be used in an immunoabsorption process, such as an ELISA, to isolate the protein or polypeptide. Tissue samples which can be assayed include mammalian tissues, e.g., differentiated and non-differentiated cells. Examples include bone marrow, thymus, kidney, liver, brain, pancreas, fibroblasts and epithelium. These antibodies are useful in diagnostic assays, or as an active ingredient in a pharmaceutical composition.

The present invention also pertains to pharmaceutical compositions comprising polypeptides and other compounds described herein. For instance, a polypeptide or protein, or prodrug thereof, of the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to well known procedures, and will depend on the ultimate pharmaceutical formulation desired. Methods of introduction of exogenous polypeptides at the site of treatment include, but are not limited to, intradermal, intramuscular, intraperitoneal, intravenous, subcutaneous, oral, rectal and intranasal. Other suitable methods of introduction can also include gene therapy, rechargeable or biodegradable devices and slow release polymeric devices. The pharmaceutical compositions of this invention can also be administered as part of a combinatorial therapy with other agents.

Screening Assays

The invention provides a method (also referred to herein as a “screening assay”) for identifying modulators, i.e., candidate or test compounds or agents (e.g., antisense, polypeptides, peptidomimetics, small molecules or other drugs) which bind to nucleic acid molecules, polypeptides or proteins described herein or have a stimulatory or inhibitory effect on, for example, expression or activity of the nucleic acid molecules, polypeptides or proteins of the invention.

In one embodiment, the invention provides assays for screening candidate or test compounds which bind to or modulate the activity of protein or polypeptide described herein or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. U.S.A., 90:6909; Erb et al. (1994) Proc. Natl. Acad. Sci. U.S.A., 91:11422; Zuckermann et al. (1994). J. Med. Chem., 37:2678; Cho et al.(1993) Science, 261:1303; Carell et al. (1994) Angew. Chem. Int. Ed. Engl., 33:2059; Carell et al. (1994) Angew. Chem. Int. Ed. Engl., 33:2061; and in Gallop et al. (1994)J. Med. Chem., 37:1233.

Libraries of compounds may be presented in solution (e.g., Houghten (1992) Biotechniques, 13:412-421), or on beads (Lam (1991) Nature, 354:82-84), chips (Fodor (1993) Nature, 364;555-556), bacteria (Ladner U.S. Pat. No. 5,223,409), spores (Ladner U.S. Pat. No. '409), plasmids (Cull et al. (1992) Proc. Natl. Acad. Sci. U.S.A., 89:1865-1869) or on phage (Scott and Smith (1990) Science, 249:386-390); (Devlin (1990) Science, 249:404-406); (Cwirla et al. (1990) Proc. Natl. Acad. Sci. U.S.A., 97:6378-6382); (Felici (1991) J. Mol. Biol., 222:301-310); (Ladner supra).

In one embodiment, an assay is a cell-based assay in which a cell which expresses an encoded protein which is contacted with a test compound and the ability of the test compound to bind to the protein is determined. The cell, for example, can be of mammalian origin, such as a thymocyte. Determining the ability of the test compound to bind to the polypeptide can be accomplished, for example, by coupling the test compound with a radioisotope, enzymatic label or fluorescent label, such that binding of the test compound to the polypeptide can be determined by detecting the labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with the polypeptide without the labeling of any of the interactants. For example, a microphysiometer can be used to detect the interaction of a test compound with the polypeptide without the labeling of either the test compound or the polypeptide. McConnell, H. M. et al. (1992) Science, 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

In another embodiment, an assay is a cell-based assay comprising contacting a cell expressing a particular target molecule described herein with a test compound and determining the ability of the test compound to modulate or alter (e.g. stimulate or inhibit) the activity of the target molecule. Determining the ability of the test compound to modulate the activity of the target molecule can be accomplished, for example, by determining the ability of the polypeptide to inhibit NFκB-induced gene expression.

In a preferred embodiment, determining the ability of the polypeptide to inhibit NFκB-induced expression can be accomplished by determining the activity of the target molecule. For example, detecting the induction of a reporter gene (comprising a target-responsive regulatory element operatively linked to a nucleic acid encoding a detectable marker, e.g., luciferase) or detecting a cellular response, for example, apoptosis, differentiation or rate of proliferation.

In yet another embodiment, an assay of the present invention is a cell-free assay in which protein of the invention or biologically active portion thereof is contacted with a test compound and the ability of the test compound to bind to the protein or biologically active portion thereof is determined. Binding of the test compound to the protein can be determined either directly or indirectly as described above. In one embodiment, the assay includes contacting the protein or biologically active portion thereof with a known compound which binds the protein to form an assay mixture, contacting the assay mixture with a test compound, and determining the ability of the test compound to interact with the protein. Determining the ability of the test compound to interact with the protein comprises determining the ability of the test compound to preferentially bind to the protein or biologically active portion thereof as compared to the known compound.

In another embodiment, the assay is a cell-free assay in which a protein of the invention or biologically active portion thereof is contacted with a test compound and the ability of the test compound to modulate or alter (e.g., stimulate or inhibit) the activity of the protein or biologically active portion thereof is determined. Determining the ability of the test compound to modulate the activity of the protein can be accomplished, for example, by determining the ability of the protein to bind to a known target molecule by one of the methods described above for determining direct binding. Determining the ability of the protein to bind to a target molecule can also be accomplished using technology such as real-time Bimolecular Interaction Analysis (BIA). Sjolander, S. and Urbaniczky, C. (1991) Anal. Chem., 63:2338-2345 and Szabo et al. (1995) Curr. Opin. Struct. Biol., 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In an alternative embodiment, determining the ability of the test compound to modulate the activity of a protein of the invention can be accomplished by determining the ability of the protein to further modulate the activity of a target molecule. For example, the activity of NFκB-induced gene expression as previously described.

In yet another embodiment, the cell-free assay involves contacting a protein of the invention or biologically active portion thereof with a known compound which binds the protein to form an assay mixture, contacting the assay mixture with a test compound, and determining the ability of the test compound to interact with the protein, wherein determining the ability of the test compound to interact with the protein comprises determining the ability of the protein to preferentially bind to or modulate the activity of a target molecule.

The cell-free assays of the present invention are amenable to use of both soluble and/or membrane-bound forms of isolated proteins. In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-14, Thesit®, Isotridecypoly (ethylene glycol ether)ₙ, 3-[(3-cholamidopropyl) dimethylamminio]-1-propane sulfonate (CHAPS), 3-[(3-cholamidopropyl) dimethylamminio]-2-hydroxy-1-propane sulfonate (CHAPSO), or N-dodecyl-N,N-dimethyl-3-ammonio-1-propane sulfonate.

In more than one embodiment of the above assay methods of the present invention, it may be desirable to immobilize either the protein or its target molecule to facilitate separation of complexed from uncomplexed forms of one or both of the proteins, as well as to accommodate automation of the assay. Binding of a test compound to the protein, or interaction of the protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or protein of the invention, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, either a protein of the invention or a target molecule can be immobilized utilizing conjugation of biotin and streptavidin. Biotinylated protein of the invention or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a protein of the invention or target molecules, but which do not interfere with binding of the protein to its target molecule, can be derivatized to the wells of the plate, and unbound target or protein trapped in the wells by antibody conjugation. Methods for detecting such complexes, in addition to those described above for the GST-immobilized complexes, include immunodetection of complexes using antibodies reactive with the protein or target molecule, as well as enzyme-linked assays which rely on detecting an enzymatic activity associated with the protein or target molecule.

In another embodiment, modulators of expression of nucleic acid molecules of the invention are identified in a method wherein a cell is contacted with a candidate compound and the expression of appropriate mRNA or protein in the cell is determined. The level of expression of appropriate mRNA or protein in the presence of the candidate compound is compared to the level of expression of mRNA or protein in the absence of the candidate compound. The candidate compound can then be identified as a modulator of expression based on this comparison. For example, when expression of mRNA or protein is greater (statistically significantly greater) in the presence of the candidate compound than in its absence, the candidate compound is identified as a stimulator or enhancer of the mRNA or protein expression. Alternatively, when expression of the mRNA or protein is less (statistically significantly less) in the presence of the candidate compound than in its absence, the candidate compound is identified as an inhibitor of the mRNA or protein expression. The level of mRNA or protein expression in the cells can be determined by methods described herein for detecting mRNA or protein.

In yet another aspect of the invention, the proteins of the invention can be used as “bait proteins” in a two-hybrid assay or three-hybrid assay (see, e.g., U.S. Pat. No. 5,283,317; Zervos et al. (1993) Cell, 72:223-232; Madura et al. (1993) J. Biol. Chem., 268:12046-12054; Bartel et al. (1993) Biotechniques, 14:920-924; Iwabuchi et al. (1993) Oncogene, 8:1693-1696; and Brent WO94/10300), to identify other proteins (captured proteins) which bind to or interact with the proteins of the invention and modulate their activity. Such captured proteins are also likely to be involved in the propagation of signals by the proteins of the invention as, for example, downstream elements of a protein-mediated signaling pathway.

The two-hybrid system is based on the modular nature of most transcription factors, which consist of separable DNA-binding and activation domains. Briefly, the assay utilizes two different DNA constructs. In one construct, the gene that codes for a protein of the invention is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected, and cell colonies containing the functional transcription factor can be isolated and used to obtain the cloned gene which encodes the protein which interacts with the protein of the invention.

This invention further pertains to novel agents identified by the above-described screening assays. Accordingly, it is within the scope of this invention to further use an agent identified as described herein in an appropriate animal model. For example, an agent identified as described herein (e.g., a modulating agent, an antisense nucleic acid molecule, a specific antibody, or a protein-binding partner) can be used in an animal model to determine the efficacy, toxicity, or side effects of treatment with such an agent. Alternatively, an agent identified as described herein can be used in an animal model to determine the mechanism of action of such an agent. In addition, this invention encompasses the use of transgenic animals that express a nucleic acid sequence selected from the group consisting of SEQ ID NOS: 1 and 3, the complement of SEQ ID NOS: 1 and 3, the nucleic acid sequence that encode SEQ ID NOS: 2 and 4.

Furthermore transgenic animals that have SEQ ID NO: 3 or fragment thereof deleted are also encompassed within this invention. These transgenic animals can be used in multiple methods to assay, for example, modulators of expression of the nucleic acid molecules of the invention identified in a method wherein the transgenic animal is exposed to a candidate compound and the expression of appropriate mRNA or protein in the cell is determined. The level of expression of appropriate mRNA or protein in the presence of the candidate compound is compared to the level of expression of mRNA or protein in the absence of the candidate compound. The candidate compound can then be identified as a modulator of expression based on this comparison. For example, when expression of mRNA or protein is greater (statistically significantly greater) in the presence of the candidate compound than in its absence, the candidate compound is identified as a stimulator or enhancer of the mRNA or protein expression. Alternatively, when expression of the mRNA or protein is less (statistically significantly less) in the presence of the candidate compound than in its absence, the candidate compound is identified as an inhibitor of the mRNA or protein expression. The level of mRNA or protein expression in the cells can be determined by methods described herein for detecting mRNA or protein and as is well known in the art.

Furthermore, this invention pertains to uses of novel agents identified by the above-described screening assays for treatments as described herein.

Methods of Treatment

The present invention provides for both prophylactic and therapeutic methods of treating a subject at risk of (or susceptible to) a disorder or having a disorder associated with aberrant expression or activity of proteins or nucleic acids of the invention.

Disorders for treatment include, but not limited to, autoimmune diseases such as arthritis (e.g., rheumatoid arthritis, juvenile rheumatoid arthritis, psoriatic arthritis), multiple sclerosis, systemic lupus erythematosus, myasthenia gravis, juvenile onset diabetes, diabetes mellitus, nephritides such as glomerulonephritis, autoimmune thyroiditis, Behcet's disease; inflammatory or allergic diseases and conditions, including respiratory allergic diseases such as asthma, allergic rhinitis, hypersensitivity lung diseases, hypersensitivity pneumonitis, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); chronic obstructive pulmonary disease; anaphylaxis or hypersensitivity responses, drug allergies (e.g., to penicillin, cephalosporins), insect sting allergies; inflammatory bowel diseases, such as Crohn's disease and ulcerative colitis; spondyloarthropathies; scleroderma; psoriasis and inflammatory dermatoses such as dermatitis, eczema, atopic dermatitis, allergic contact dermatitis, urticaria; vasculitis (e.g., necrotizing, cutaneous, and hypersensitivity vasculitis), graft rejection (e.g., in transplantation), including allograft rejection or graft-versus-host disease, and organ transplant-associated arteriosclerosis; cancer, tuberculosis, malaria and HIV infection.

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with the molecules of the present invention or modulators according to that individual's drug response genotype. Pharmacogenomics allows a clinician or physician to target prophylactic or therapeutic treatments to patients who will most benefit from the treatment and to avoid treatment of patients who will experience toxic drug related side effects.

1. Prophylactic Methods

In one aspect, the invention provides a method for preventing in a subject, a disease or condition associated with aberrant expression or activity of genes or proteins of the present invention, by administering to the subject an agent which modulates expression or at least one activity of a gene or protein of the invention. Subjects at risk for a disease which is caused or contributed to by aberrant gene expression or protein activity can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein. The prophylactic methods of the present invention are further discussed in the following subsections.

2. Therapeutic Methods

Another aspect of the invention pertains to methods of modulating expression or activity of genes or proteins of the invention for therapeutic purposes. The modulatory method of the invention involves contacting a cell with an agent that modulates one or more of the activities of the specified protein associated with the cell. An agent that modulates protein activity can be an agent as described herein, such as a nucleic acid or a protein, a naturally-occurring target molecule of a protein described herein, a polypeptide, a peptidomimetic, or other small molecule. In one embodiment, the agent stimulates one or more protein activities. Examples of such stimulatory agents include active protein as well as a nucleic acid molecule encoding the protein that has been introduced into the cell. In another embodiment, the agent inhibits one or more protein activities. Examples of such inhibitory agents include antisense nucleic acid molecules and anti-protein antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a protein or nucleic acid molecule of the invention. In one embodiment, the method involves administering an agent (e.g., an agent identified by a screening assay described herein), or combination of agents that modulates (e.g., upregulates or downregulates) expression or activity of a gene or protein of the invention. In another embodiment, the method involves administering a protein or nucleic acid molecule of the invention as therapy to compensate for reduced or aberrant expression or activity of the protein or nucleic acid molecule.

Stimulation of protein activity is desirable in situations in which the protein is abnormally downregulated and/or in which increased protein activity is likely to have a beneficial effect. Likewise, inhibition of protein activity is desirable in situations in which the protein is abnormally upregulated and/or in which decreased protein activity is likely to have a beneficial effect. One example of such a situation is where a subject has a disorder characterized by aberrant development or cellular differentiation. Another example of such a situation is where the subject has a proliferative disease (e.g., cancer) or a disorder characterized by an aberrant hematopoietic response.

3. Pharmacogenomics

The molecules of the present invention, as well as agents, or modulators which have a stimulatory or inhibitory effect on the protein activity (e.g., gene expression) as identified by a screening assay described herein can be administered to individuals to treat (prophylactically or therapeutically) disorders (e.g., proliferative or developmental disorders) associated with aberrant protein activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a molecule of the invention or modulator thereof, as well as tailoring the dosage and/or therapeutic regimen of treatment with such a molecule or modulator.

Pharmacogenomics deals with clinically significant hereditary variations in the response to drugs due to altered drug disposition and abnormal action in affected persons. See e.g., Eichelbaum, M., Clin Exp Pharmacol. Physiol., (1996) 23(10-11):983-985 and Linder, M. W., Clin. Chem. (1997) 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1,000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a protein or a receptor of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2(NAT 2) and cytochrome P450 enzymes CYP2D6 and CYP2C19) has provided an explanation as to why some patients do not obtain the expected drug effects or show exaggerated drug response and serious toxicity afer taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a molecule or modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a molecule or modulator of the invention, such as a modulator identified by one of the exemplary screening assays described herein.

### EXAMPLES

Materials and Methods

Animals

N15tg RAG-2^{-/-} TCR H-2^{b} and N15tg RAG-2^{-/-} TCR H-2^{d} mice were generated as described (Ghendler et al., 1998). C57BL/6 and TCRCyt5CC7-I-RAG-2^{-/-} mice were obtained from Taconic. Mice were maintained and bred under sterile barrier conditions at the animal facility of the Dana-Farber Cancer Institute.

Representational Difference Analysis (RDA)

Thymii were dissected from 3 week old N15tg RAG-2^{-/-} TCR H-2^{b} mice 1 h after i.v. injection of 24 μg VSV8 peptide or from untreated 3 week old N15tg RAG-2^{-/-} TCR H-2^{d} mice. The thymii were dissociated in PBS-1% BSA thymocytes washed in PBS-2% FCS and stained with anti-CD4 mAb (BD Pharmingen). In these class I-restricted transgenic animals, all CD4+ cells are DP. DP thymocytes were sorted on a MoFlo (Cytomation) into ice cold PBS-2% FCS and RNA prepared using guanidium isothiocyanate as described (Lerner et al., 1996). The RNA was polyA-selected using the polyA Spin mRNA Isolation Kit (New England Biolabs). This mRNA was then used for RDA (Lisitsyn et al., 1993; Hubank and Schatz, 1994). Due to limitations in the amount of mRNA, a subtracted library in the pZErO-1 vector (Invitrogen) using difference product 2 was prepared instead of difference product 3. For this purpose, difference product 2 was size fractionated on an agarose gel; 6 gel slices containing DNAs of different sized-fragments were eluted and ligated to pZErO-1. DNA from one gel slice containing an obvious PCR band was ligated, transformed and screened with a full length nur77 probe. DNA was prepared from nur77 negative clones for sequencing and use as probes in Northern blot analysis.

Constructs

Full length IκBNS was obtained by screening a cDNA library in λZAP Express (Stratagene) prepared from the thymii of C57BL/6 mice sacrificed 2, 4 and 6 h after injection of 200 μg anti-CD3ε mAb. After in vivo excision, the cDNA was in the pBKCMV vector. For detection of protein upon transfection into Cos-7 cells, a Flag tag was added to IκBNS by PCR and the product subcloned into pCR2.1 using the TA Cloning Kit (Invitrogen) and then into pcDNA3.1 (Invitrogen) with KpnI and EcoRV. To produce a fusion protein with GST, a BamHI/XhoI fragment from Flag-tagged pCR2.1 IκBNS was ligated to BamHI/XhoI-cut pGEX-4T-1 (Amersham Pharmacia Biotech). GST-IκBNS was purified following the procedures recommended by Amersham Pharmacia Biotech. The superinhibitor IκBα was used to transfect Cos-7 cells and to produce a GST fusion protein. For this purpose, IκBα from CD2 MAD (Hettmann et al., 1999) was PCR'd and subcloned into pCR2.1 using the TA Cloning Kit (Invitrogen) and the EcoRI/SalI fragment subcloned into EcoRI/XhoI cut pcDNA3.1. For GST-IκBα, the EcoRI/SalI fragment of pCR2.1-IκBα was ligated to EcoRI/SalI-cut pGEX-4T-1. GST-IκBα protein was purified as described above.

FTOC

For FTOC, pregnant N15tg RAG-2^{-/-} TCR H-2^{b} mice were sacrificed and fetal thymii removed at day 15.5 with the observation of vaginal plug as day 1. Fetal thymii were cultured in AIM-V media (Invitrogen) in Transwell dishes (Costar) in a humidified atmosphere with 5% CO₂ for 5 days. On day 5, peptides were added to 10 μM. Thymocytes were harvested 2 h after peptide addition and RNA prepared using the Qiagen RNeasy Mini kit according to the manufacturer's protocol (Qiagen).

Antisense Oligonucleotide Blockade

FTOC were set up as described. On day 4, phosphorothioate oligonucleotides (sense, 5′CCCCTGGTGATGGAGGACTCT3′, or antisense, 5′AGAGTCCTCCATCACCAGGGG3′ from MWG Biotech, Inc.) were added at 200 μg/ml. After 12-19 h, VSV8 peptide was added to some FTOC at 300 μM. After 4 more hours, thymic lobes were harvested and analyzed by FACS. Thymocytes were stained at ˜5×10⁶ cells per ml in PBS-2% FCS-0.05% NaN3 containing the antibodies at saturating concentrations. The antibodies were anti-CD8α-FITC (53-6.7) and anti-CD4-PE (RM4.5) from Pharmingen. The phenotypes and proportions of thymocyte subsets were analyzed by two-color flow cytometry using a FACScan (Becton Dickinson) and the CellQuest program. Dead cells were excluded by gating.

Northern Analysis

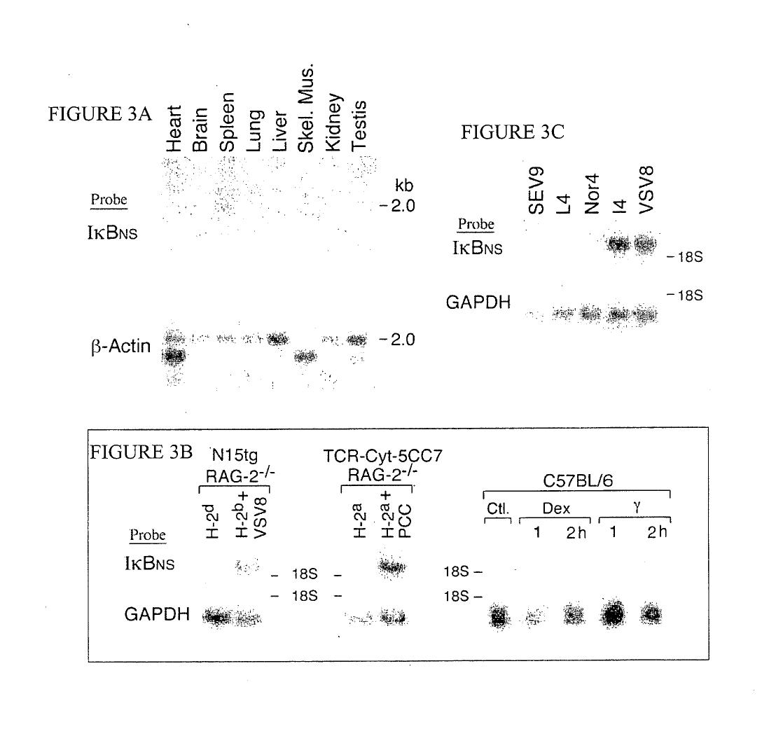

Total thymic RNA was prepared from mice or cultures treated as indicated in FIGS. 3B and 3C using guanidium isothiocyanate (Lerner et al., 1996). FTOC RNA was prepared as described above. RNA was eletrophoresed on 1% agarose/formaldehyde gels and transferred to Biotrans nylon membranes (ICN). Blots were hybridized to 1×10⁶ cpm/ml probe at 65° C. in 7% SDS/0.5M NaPO4 pH 7.2/1% BSA, washed and autoradiographed. Probes were labeled using the Random Prime Labeling Kit (Roche Biochemicals).

Peptides

SEV9, PCC, VSV8 and VSV8 variant peptides were synthesized by standard solid phase methods on an Applied Biosystems 430A synthesizer (Sasada et al., 2000). For in vivo injections, 24 μg peptide in PBS was injected intravenously into mice and the mice sacrificed at the indicated times.

Transfections and Luciferase Assays

Cos-7 cells were transfected using the calcium phosphate method (Turner et al., 1990). Cos-7 cells were plated at 0.5×10⁶ cells/well in 6 well dishes and transfected the following day with 0.2 μg pRL-null (Promega) plus 5 μg (kB)₃ luciferase plasmid (Plaisance et al., 1997). In addition, 10 μg of pcDNA3.1 or pcDNA3.1-IκBNS or pcDNA3.1-IκBα were co-transfected with the two luciferase plasmids. 24 h after transfection cells were left untreated or activated with 50 ng/ml PMA for 7 h. Cell lysates were then prepared using the Dual-Luciferase Reporter Assay System (Promega) and luciferase activity measured on a Monolight 2010 luminometer (Analytical Luminescence Laboratory). The pRL-null vector served as an internal control for transfection (Behre et al., 1999).

Electrophoretic Mobility Shift Assays

Nuclear and cytoplasmic extracts were prepared as previously described (Schreiber et al., 1989) from the thymus of C57BL/6 mice or N15tg RAG-2^{-/-} TCR H-2^{b} mice treated as described in the text. Protein concentrations were determined by Coomassie Protein Assay Reagent. NF-κB (MWG-Biotech, Inc.) and AP-1 (Santa Cruz Biotechnology) double stranded probes were kinased and used to determine binding activity. 4 μg nuclear extracts were incubated for 30 min at room temperature with 1×10⁴ counts of probe in 20 μl binding buffer (20 mM Tris pH 7.5, 0.1M NaCl, 2 mM DTT, 1 mM EDTA, 1 mg/ml BSA, 0.1% NP-40, 4% glycerol) containing 1 μg poly(dIdC) (Amersham Pharmacia Biotech). DNA-protein complexes were resolved by electrophoresis on 5% (30:1.2; acrylamide:bis) polyacrylamide gels in 0.5×TBE. The gels were then dried and autoradiographed. Where indicated, 1 μg GST, GST-IκBNS, or GST-IκBα were added to the thymic lysates and incubated 30 min on ice prior to addition of the probe.

Protein Interaction Assays

To examine the association of IκBNS with NF-κB family members, murine NF-κB family members p50 and p65, separately or together, were in vitro translated in the presence of [³⁵S]cysteine/methionine (NEN Life Science Products) using TNT-coupled reticulocyte lysate systems (Promega). In vitro-translated proteins were incubated with 5 μg of GST fusion proteins precoupled to Glutathione Sepharose 4B beads (Amersham Pharmacia Biotech) for 2 h at 4° C. in 0.5 ml incubation buffer (PBS/0.1% Triton X-100/1 mM PMSF). After washing, associated proteins were analyzed by 10% SDS-PAGE followed by autoradiography. For analysis of IκBNS-interacting NF-κB proteins in the thymus, mice were injected i.v. with VSV8 peptide or left untreated and subsequently sacrificed at various time points. Single cell suspensions of thymocytes were prepared as described above (RDA). For cytosolic extracts, thymocytes were resuspended in 250 μl of buffer A (10 mM Hepes pH 7.9/1.5 mM MgCl2/10 mM KCl/0.5 mM DTT/0.2 mM PMSF), incubated for 10 min on ice and centrifuged (8000 rpm, 8 min, 4° C.). Supernatants were collected as cytosolic extracts while pellets were resuspended in 250 μL of buffer C (20 mM Hepes pH 7.9/25% Glycerol/420 mM NaCl/1,5 mM MgCl2/0.2 mM EDTA/0.5 mM DTT/0.2 mM PMSF/10 μg/ml each of aprotinin and leupeptin). After incubation for 20 min on ice, extracts were centrifuged (14000 rpm, 15 min, 4° C.) and supernatants were collected as nuclear extracts. Protein concentrations were determined using the Micro BCA Protein Assay Reagent Kit. Extracts were adjusted to 20 mM Hepes pH 7.9/150 mM NaCl/0.1% Triton X-100/1 mM PMSF (final volume 750 μl) and precleared with Glutathione Sepharose 4B beads for 1 h at 4° C. Subsequently, extracts were incubated with GST-, GST-IκBNS-, and GST-IκBα-beads for 1 h at 4° C. Finally, beads were washed with 20 mM Hepes pH 7.9/150 mM NaCl/0.1% Triton X-100/1 mM PMSF, boiled in standard reducing sample buffer and analyzed by Western blot with the indicated antibodies (p52, p65, c-Rel and RelB antibodies from Santa Cruz; p50 antibody from Stressgen).

### Example 1

cDNA Cloning of IκBNS

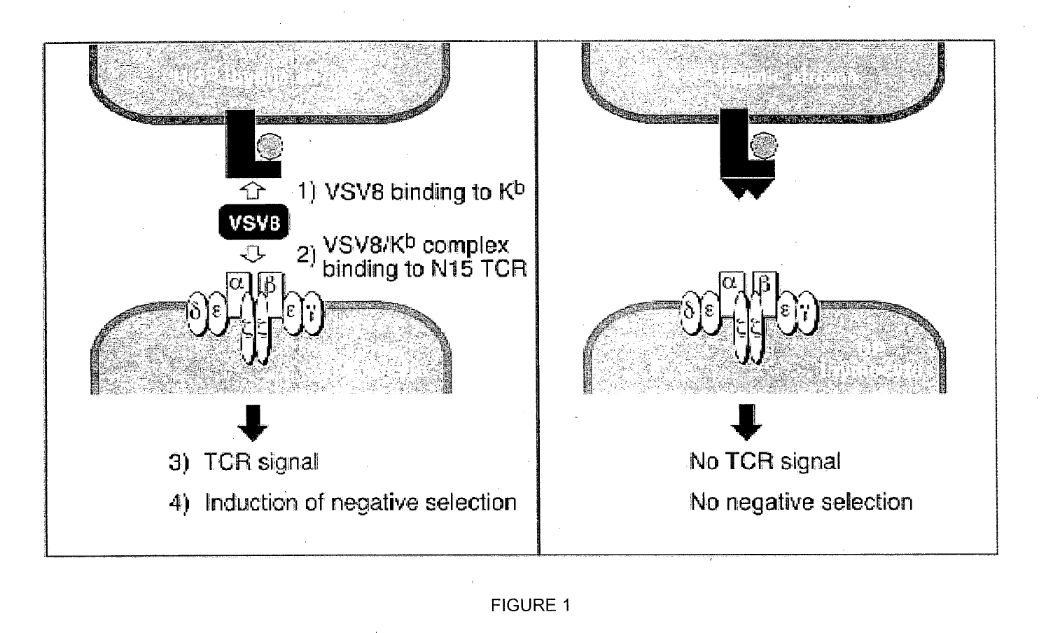

Homozygous N15 TCRtg RAG-2^{-/-} H-2^{b} mice bearing a TCR specific for the VSV8 peptide bound to the H-2 K^{b} MHC class I molecule were previously established to investigate processes of negative selection (Clayton et al., 1997; Ghendler et al., 1998; Sasada et al., 2000). Given that these mice express only a single TCR, the consequences of parenteral injection of the VSV8 peptide on thymocyte fate at various stages of development could be readily examined. These studies showed that DP thymocytes underwent apoptosis within 3 h of a single 24 μg VSV8 administration (Clayton et al., 1997). Consequently, for cDNA cloning purposes, DP thymocytes were isolated by fluorescence activated cell sorting from the thymii of N15tg RAG-2^{-/-} TCR transgenic mice on an H-2^{b} or H-2^{d} MHC background. The N15tg RAG-2^{-/-} H-2^{b} mice were injected 1 h previously with VSV8 peptide to induce negative selection. The resulting RNAs from DP thymocytes were used to perform RDA (Lisitsyn et al., 1993; Hubank and Schatz, 1994) with the VSV8-stimulated N15tg RAG-2^{-/-} H-2^{b} RNA as the tester and the unstimulated N15tg RAG-2^{-/-} H-2^{d} RNA as the driver. The mRNA pool from the N15tg RAG-2^{-/-} H-2^{d} mice is representative of DP thymocyte transcripts, but importantly, lacks those mRNAs induced via TCR activation since the N 15 TCR cannot be triggered on the H-2^{d} background (FIG. 1). The mRNA pool of the N15tg RAG-2^{-/-}H-2^{b} mice 1 h after VSV8 peptide injection contains the same DP thymocyte mRNAs and, in addition, mRNAs induced during TCR-triggered negative selection. Thus, RDA using these two DP thymocyte populations will preferentially amplify cDNA copies of mRNAs induced during the process of negative selection. The use of an early time point and sorted thymocytes obviates any RNA contribution from thymic stromal elements activated as a consequence of molecular crosstalk between thymocytes and nonlymphoid components as observed previously (Lerner et al., 1996).

PCR products were size fractionated and subcloned to create a subtracted library. Of these inserts, 80% hybridized to nur77 and were excluded from further analysis. Plasmid DNAs were prepared from randomly chosen colonies of the remaining 20% and inserts used to probe Northern blots of total thymus RNA from N15tg RAG-2^{-/-} H-₂^{d} and VSV8-injected N15tg RAG-2^{-/-} H-2^{b} mice. One insert of three tested showed strong induction in the VSV8-injected N15tg RAG-2^{-/-} H-₂^{b} RNA but did not hybridize to the N15tg RAG-2^{-/-} H-2^{d} RNA (see below). Re-analysis of the nur77-unrelated clones in the subtracted library showed that half hybridized with this novel cDNA. For further characterization, the 176 bp insert was used to screen a thymic cDNA library and the full length clone obtained.

### Example 2

Comparison with Known IκB Proteins

The predicted protein sequence (SEQ ID NO: 4) of the full-length cDNA product (SEQ ID NO: 3) is shown in FIG. 2. This sequence is highly homologous to members of the IκB family of proteins as aligned in FIG. 2. Therefore, this gene product has been termed IκBNS. The clone encodes a 327 amino acid protein containing 7 ankyrin domains (labeled A-G, FIG. 2), 4 potential protein kinase C phosphorylation sites ([S/T]-X-[R/K] at aa 20-22, 43-45, 220-222 and 289-291) and one potential casein kinase II phosphorylation site ([S/T]-X(2)-[D/E] at aa 214-217). IκBNS does not contain a PEST sequence as is found in IκBα and involved in the control the basal turnover of IκBα protein levels (Verma et al., 1995). The IκBNS protein sequence has highest similarity (43%) with “molecule possessing ankyrin-repeats induced by lipopolysaccharide” (MAIL or IκBζ), an LPS-inducible IκB protein (Kitamura et al., 2000; Yamazaki et al., 2001). Other IκB family members have 29-39% similarity by BLAST search. The FIG. 2 dendrogram shows that next to MAIL (IκBζ), human Bcl-3 is most similar. Thus, a new IκB family member has been identified.

A comparison of the ankyrin domains of six IκB family members is presented in FIG. 2. Ankyrin repeats span over 33 residues and are highly divergent (Bork, 1993). However, a basic core motif consisting of two antiparallel α-helices connected by a tight-hairpin loop is a conserved key feature. Tandem arrays of ankyrin repeats give rise to right-handed superhelixes, where one helix of the core motif (inner helix) interacts with the next inner helix of the following repeat (Gorina and Pavletich, 1996; Huxford et al., 1998; Jacobs and Harrison, 1998; Mandiya et al., 1999; Zhang and Peng, 2000). When an alignment using the entire IκBNS protein sequence is performed, the first 6 of 7 ankyrin domains in IκBNS align with ankyrin domains 1 through of the structurally characterized IκB protein, IκBα (1nfi) (Jacobs and Harrison, 1998). Repeats B, C, E, F are the most conserved whereas the N-terminal (A) and C-terminal (G) repeats are much less conserved. Ankyrin repeats do not behave as independent folded units but associate in tandem to provide stabilizing interactions (McDonald and Peters, 1998; Zhang and Peng, 2000). In this context, the C-terminal and N-terminal ankyrin repeats interact only with one neighboring repeat, and therefore have less evolutionary constraints imposed upon them. Consequently, these terminal repeats are more prone to amino acid sequence substitutions.

Ankyrin repeat D of IκBNS deserves special attention. IκBNS and MAIL (IκBζ) have an insertion in ankyrin repeat D that lies within the tight β-turn of the ankyrin repeats of p100, p105, Bcl-3 and IκBα (FIG. 2). Secondary structure predictions indicate that IκBNS has an extended outer helix rather than a long loop insertion between the inner and outer helices of the ankyrin repeat. If this is the case, the packing of the ankyrin repeats would be interrupted at this point, and a second tandem of ankyrin repeats would then start at ankyrin repeat E and terminate with ankyrin repeat G.

Control of IκBα protein levels is mediated through phosphorylation-ubiquitination processes resulting in its degradation. The DSGL[D/G/EI]S (SEQ ID NO:5) motif found in IκBα, β and ε is not present in IκBNS. Furthermore, there are no lysines equivalent to those in IκBa which can serve as targets for ubiquitination (Karin and Ben-Neriah, 2000) implying that IκBNS is insensitive to this degradation pathway. In addition, control of the basal IκBα protein level is through a PEST sequence located in the carboxy terminal portion (Karin and Ben-Neriah, 2000) and not found in IκBNS.

### Example 3

Human Homolog

A blast search using the 176 bp insert of the original RDA clone revealed high similarity with a human genomic sequence in Genbank. This sequence consists of 39,163 bp from human chromosome 19 and contains APLP1, a transmembrane glycoprotein related to Alzheimer disease associated amyloid beta protein precursor. The full length clone of IκBNS also matches this human chromosome 19 sequence in discontinuous stretches over a large area (from ˜bp 23,000 to bp 33,500 in the antisense direction relative to APLP1) suggesting the presence of a homologous human gene with 7 or more exons, (SEQ ID NO: 1). Comparison of the murine IκBNS protein sequence (SEQ ID NO: 4) with that of the hypothetical (predicted) human protein (SEQ ID NO: 2) shows 91% identity. Hence, a human IκBNS gene exists and likely serves a comparable function to the mouse counterpart.

### Example 4

Analysis of IκBNS mRNA Expression

Using the 176 bp RDA product as a probe, we examined the tissue distribution of this gene as shown in FIG. 3A. While faint hybridization to spleen mRNA was noted, heart, brain, lung, liver, skeletal muscle, kidney and testis were negative. In addition, very low levels can be detected in RNA from C57BL/6 thymus (FIG. 3B, right panel) perhaps as a consequence of ongoing negative selection. However, in N15tg RAG-2^{-/-} H-2^{d} or N15tg RAG-2^{-/-} H-2^{b} animals, IκBNS message is not detected in the thymus of control animals but is apparent within 1 h of i.v. injection of VSV8 peptide into N15tg RAG-2^{-/-} H-2^{b} mice (FIG. 3B, left panel). Similarly, IκBNS message is strongly induced within 1 h of pigeon cytochrome c (PCC) peptid (aa 88-104) injection into TCRCyt5CC7-I-RAG-2^{-/-} H-2^{a} mice which bear a TCR recognizing PCC bound to the I-Ek class II MHC molecule (Seder et al., 1992) (FIG. 3B, middle panel). Under these conditions PCC peptide administration induces negative selection of DP thymocytes in TCRCyt5CC7-I-RAG-2^{-/-} H-2^{a} mice. Note the absence of IκBNS message in the basal state. Collectively, the results demonstrate that induction of negative selection in both class I— and class II-restricted TCR transgenic animals results in increased levels of IκBNS mRNA. This message is also induced in thymic RNA isolated from C57BL/6 mice treated in vivo with anti-CD3E monoclonal antibody, a treatment known to induce death in DP thymocytes (Smith et al., 1989). While apoptotic death of immature thymocytes is also induced by glucocorticoids (Wyllie, 1980) and γ-irradiation (Sellins and Cohen, 1987), thymic RNA from mice treated with 0.5 mg dexamethasone or 500 rads show essentially no induction of IκBNS when mRNA levels are normalized with GAPDH message (FIG. 3B). Thus IκBNS is induced only by TCR-triggered events in immature thymocytes

Analyses of the N15tg RAG-2^{-/-} H-2^{b} mice using variant peptide ligands have demonstrated that the VSV8 peptide p4 position is critical in determining thymic selection outcomes (Ghendler et al., 1998; Sasada et al., 2000). Hence, small changes in the peptide there can be sensed by the TCR. While VSV8 or the isoleucine variant (14) induce negative selection in FTOC using N15tg RAG-2^{-/-} H-2^{b} mice, leucine (L4) or norvaline (Nor4) substitutions result in positive selection (Clayton et al., 1997; Ghendler et al., 1998; Sasada et al., 2000). Using this information, induction of IκBNS in N15tg RAG-2^{-/-} H-2^{b} FTOC treated with these peptides was analyzed. SEV9, a Sendai virus-derived peptide that binds Kb with an affinity equal to that of VSV8 but is not recognized by the N15 TCR serves as a control peptide. As shown in FIG. 3C, the negatively selecting peptides, VSV8 and 14, result in IκBNS mRNA induction within 2 h of addition to N15tg RAG-2^{-/-} H-2^{b} FTOC. In contrast, the L4 and Nor4 peptides that induce positive selection and the control non-selecting SEV9 peptide do not upregulate steady state expression of the IκBNS mRNA. Induction of IκBNS mRNA therefore correlates with the process of negative selection in thymocytes.

### Example 5

NF-κB Inhibition by IκBNS

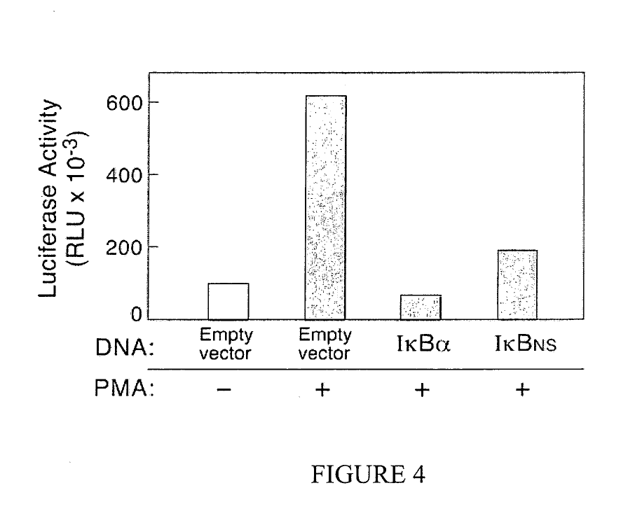

The major function of IκBα is to bind NF-κB in the cytoplasm and prevent its nuclear translocation and subsequent DNA binding by blocking these sites on NF-κB. To determine if IκBNS has a similar inhibitory function, an NF-κB-sensitive luciferase reporter in Cos-7 cells cotransfected with IκBNS was assayed. NF-κB activity was induced by a 7 h treatment of transfected Cos-7 cells with 50 ng/ml phorbol-12-myristate-13-acetate (PMA) prior to analysis of luciferase activity. PMA treated Cos-7 cells showed an increase of approximately 5-10 fold in NF-κB luciferase activity relative to untreated cells (FIG. 4). Cotransfection with an IκBNS construct reduced NF-κB-driven luciferase activity to a level approaching that of cells transfected with the vector only. Cotransfection of IκBα with the NF-κB luciferase construct inhibited PMA-induced NF-κB luciferase activity almost completely. Thus both IκBNS and IκBα reduce NF-κB-sensitive reporter activity in Cos cells. IκBNS also reduced the activity of NF-κB-sensitive reporter constructs in transiently transfected Jurkat T cells.

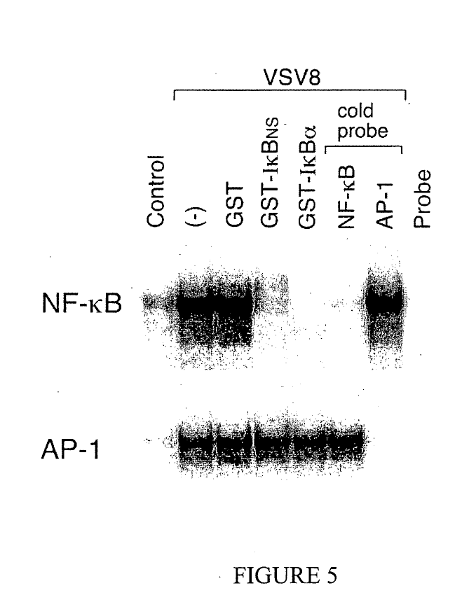

To further assay IκBNS function, the effects of a chimeric GST-IκBNS fusion protein on NF-κB electrophoretic mobility shift assays (EMSAs) was tested. NF-κB gel shift analysis was performed on nuclear extracts from N15tg RAG-2^{-/-} H-2^{b} mice 1 h after VSV8 injection (FIG. 5). Addition of GST-IκBNS and GST-IκBa but not GST blocks the gel shift band in N15tg RAG-2^{-/-} H-2^{b} thymic nuclear extracts. GST-IκBNS also blocks NF-κB binding in nuclear lysates of anti-CD3ε mAb-treated C57BL/6 animals. Addition of 100×cold NF-κB probe blocks formation of the NF-κB band but 100×cold AP-1 probe has no effect, demonstrating the specificity of this gel shift. In contrast, the GST-fusion proteins have no affect on the AP-1 gel shift, but the 100×cold AP-I probe inhibits formation of this band. Collectively, these data suggest that IκBNS and IκBα bind the NF-κB components and prevent subsequent DNA binding.

### Example 6

Interaction of IκBNS with NF-κB Family Members

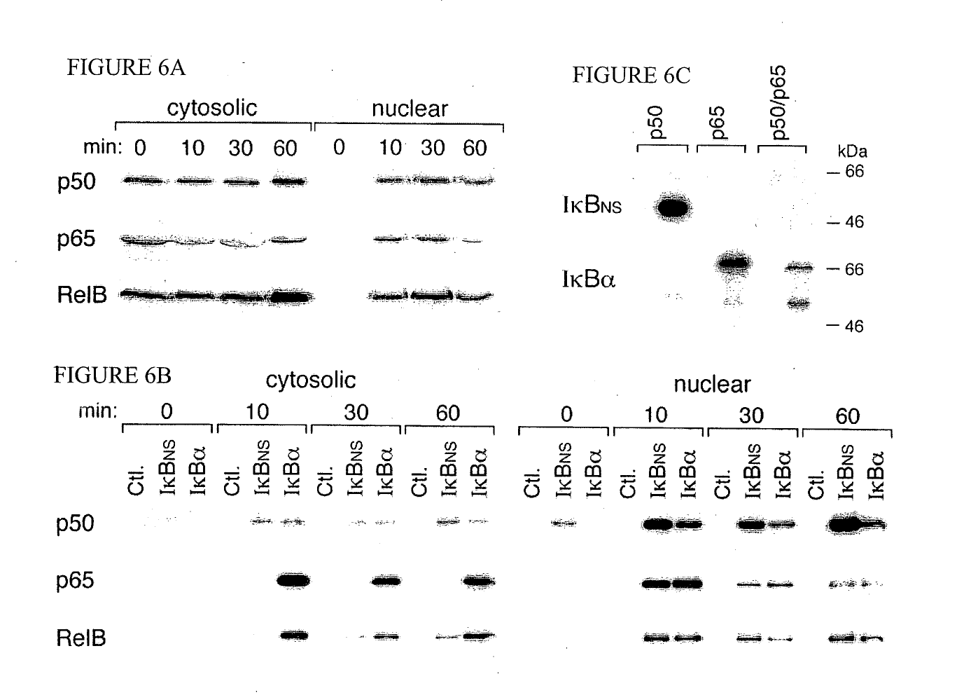

To examine the specificity of IκBNS binding in vivo, thymic cytosolic and nuclear lysates were prepared from N15tg RAG-2^{-/-} H-₂^{b} mice 10, 30 or 60 min after i.v. VSV8 injection or from uninjected animals and used for Western blot analysis of IκBNS- and IκBa-interacting proteins. The total cellular levels of p50, p65 and RelB proteins are shown in FIG. 6A. There is little change in their cytosolic level upon VSV8 injection; in contrast, p50, p65 and RelB proteins are barely detectable in the nuclear fraction in control animals. Within 10 min of TCR triggering, however, all are readily observed in the nucleus. Presumably triggering through the TCR has induced degradation of cytosolic IκBα and allowed translocation of these proteins to the nucleus. Both c-Rel and p52 show similar patterns of expression to those in FIG. 6A although p52 is detectable in the nuclear fraction of the 0 time point.

GST, GST-IκBNS and GST-IκBα were incubated with the thymic lysates and interacting proteins identified by Western blot analysis (FIG. 6B). GST protein (Ctl) does not interact with any of the proteins analyzed. Aside from some basal p50-IκBNS interaction, the interaction of GST-IκBNS and GST-IκBa with p50, p65 and RelB proteins is largely dependent on TCR triggering. The presence of endogenous cytosolic IκBα at the 0 time point likely blocks interaction with the GST-Iκ B α and GST-IκBNS fusion proteins. In the cytosolic fraction, interaction with p50 and RelB are readily detected in the IκBNS pull down experiment at 10, 30 and 60 min. However, no p65 association is detected at any time point tested even though p65 is present in this fraction (FIG. 6A) and can interact with IκBα (FIG. 6B). The preference of IκBNS for p50 and IκBα for p65 is born out in interaction analysis on in vitro translated proteins (FIG. 6C). Moreover, GST-IκBα binds all three proteins in the cytosol consistent with its ability to interact with various NF-κB heterodimers. The amount of cytosolic p50 bound to GST-IκBNS and GST-IκBα is approximately equivalent while more cytosolic RelB is bound by GST-IκBα than by GST-IκBNS. The pattern of interaction with cytosolic c-Rel is exactly like that of RelB for both GST-IκBNS and GST-IκBα.

Within the nuclear fraction, however, GST-IκBNS displays a surprisingly similar pattern of NF-κB binding to that of GST-IκBα, interacting with p50, p65 and RelB although binding more p50. While the amounts of total p50, p65 and RelB proteins are approximately equal in both nuclear and cytoplasmic compartments (FIG. 6A) (aside from the 0 time point where no p65 or RelB is detected in the nuclear fraction of control mice), IκBNS binding is more apparent in the nuclear than cytosolic fractions. GST-IκBNS binds to p52 only in the nuclear fraction and the pattern of interaction with c-Rel is exactly like that of RelB. That the GST-IκBNS interaction differs between the nuclear and cytosolic fractions is noteworthy. In the nucleus NF-κB proteins may be bound to DNA and, hence, the conformation in the presence of DNA altered to allow interaction with IκBNS in the nuclear fraction. A second possibility is that there may be TCR-triggering-induced modification of NF-κB proteins that allows an interaction with GST-IκBNS in the nucleus. A third possibility might involve a distinct protein(s) within the nucleus that facilitates the interaction of IκBNS with p65 and RelB (and c-Rel) in this compartment.

### Example 7

IκBNS Blockade Inhibits Antigen-Induced Negative Selection

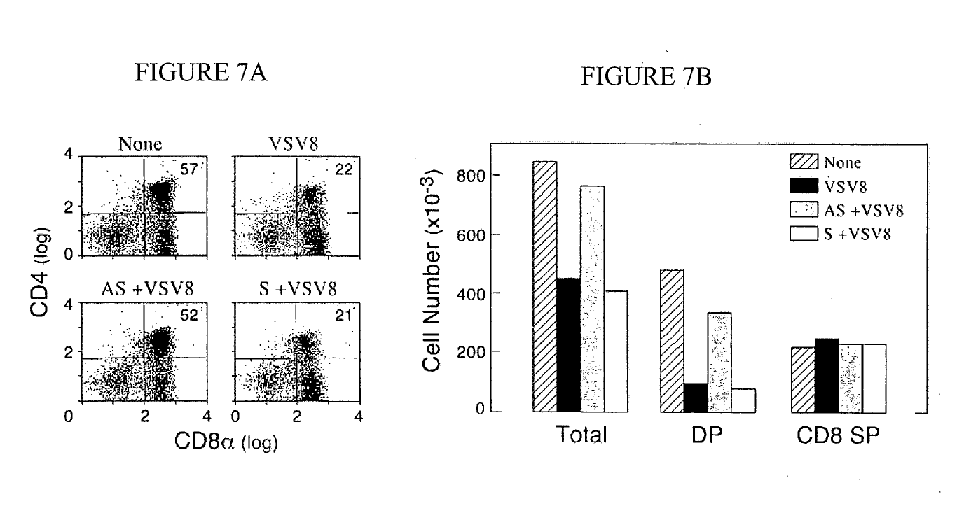

To determine the functional consequence of inhibition of IκBNS expression during antigen-induced TCR triggering of DP thymocytes, antisense oligonucleotides to block IκBNS translation in FTOC of N15tg RAG-2^{-/-} H-2^{b} mice were used. A 21 bp phosphorothioate antisense oligonucleotide was designed to hybridize to the initiating ATG of IκBNS as well as 9 bases in the 5′ and 3′ directions (Hattori et al., 1996). An oligonucleotide consisting of the complimentary 21 bases in the sense direction was used as a control. On the fourth day of culture, sense and antisense oligonucleotides were added to FTOCs at 200 μg/ml. After 12-19 h, VSV8 peptide was added to some FTOC and the culture continued for 4 h. As shown in FIG. 7A, the control FTOC contains approximately 57% DP thymocytes. Four hours after VSV8 treatment, the percentage of DP thymocytes is reduced to 22%. The presence of the IκBNS antisense oligonucleotide (AS) prevents deletion of DP cells (52% DP), while the IκBNS sense oligonucleotide (S) has no effect (21% DP). Although not shown, IκBNS antisense or sense oligonucleotides in the absence of VSV8 had no effect on the percentage of DP thymocytes in FTOC. Changes in the absolute numbers of total thymocytes as well as DP and CD8 SP subpopulations from these FTOC are shown in FIG. 7B. VSV8 induces a 50% reduction in the total number of thymocytes; this loss is confined to the DP thymocyte compartment as the number of CD8 SP thymocytes remains unchanged. Strikingly, the presence of the antisense IκBNS oligonucleotide almost completely blocks the antigen-induced loss of thymocytes while the sense oligonucleotide has no effect.

### REFERENCES

Alam, S. M., Travers, P. J., Wung, J. L., Nasholds, W., Redpath, S., Jameson, S. C. and Gascoigne, N. R. (1996) T-cell-receptor affinity and thymocyte positive selection. Nature, 381, 616-620.
Amsen, D., Revilla Calvo, C., Osborne, B. A. and Kruisbeek, A. M. (1999) Costimulatory signals are required for induction of transcription factor Nur77 during negative selection of CD4(+)CD8(+) thymocytes. Proc Natl Acad Sci U S A, 96, 622-7.
Baeuerle, P. A. (1998) IkappaB-NF-kappaB structures: at the interface of inflammation control. Cell, 95, 729-31.
Bakker, T. R., Renno, T. and Jongeneel, C. V. (1999) Impaired fetal thymocyte development after efficient adenovirus-mediated inhibition of NF-kappa B activation. J Immunol, 162, 3456-62.
Behre, G., Smith, L. T. and Tenen, D. G. (1999) Use of a promoterless Renilla luciferase vector as an internal control plasmid for transient co-transfection assays of Ras-mediated transcription activation. Biotechniques, 26, 24-6, 28.
Bork, P. (1993) Hundreds of ankyrin-like repeats in functionally diverse proteins: mobile modules that cross phyla horizontally? Proteins, 17, 363-74.
Bours, V., Franzoso, G., Azarenko, V., Park, S., Kanno, T., Brown, K. and Siebenlist, U. (1993) The oncoprotein Bcl-3 directly transactivates through kappa B motifs via association with DNA-binding p50B homodimers. Cell, 72, 729-39.
Clayton, L. K., Ghendler, Y., Mizoguchi, E., Patch, R. J., Ocain, T. D., Orth, K., Bhan, A. K., Dixit, V. M. and Reinherz, E. L. (1997) T-cell receptor ligation by peptide/MHC induces activation of a caspase in immature thymocytes: the molecular basis of negative selection. EMBO J, 16, 2282-2293.
Cohen, J. J., Duke, R. C., Fadok, V. A. and Sellins, K. S. (1992) Apoptosis and programmed cell death in immunity. Annu Rev Immunol, 10, 267-93.
D'Adamio, L., Clayton, L. K., Awad, K. M. and Reinherz, E. L. (1992) Negative selection of thymocytes. A novel polymerase chain reaction-based molecular analysis detects requirements for macromolecular synthesis. J Immunol, 149, 3550-3.
Davis, M. M. and Bjorkman, P. J. (1988) T-cell antigen receptor genes and T-cell recognition. Nature, 334, 395-402.
Dechend, R., Hirano, F., Lehmann, K., Heissmeyer, V., Ansieau, S., Wulczyn, F. G., Scheidereit, C. and Leutz, A. (1999) The Bcl-3 oncoprotein acts as a bridging factor between NfkappaB/Rel and nuclear co-regulators. Oncogene, 18, 3316-23.
Ferreira, V., Sidenius, N., Tarantino, N., Hubert, P., Chatenoud, L., Blasi, F. and Korner, M. (1999) In vivo inhibition of NF-kappa B in T-lineage cells leads to a dramatic decrease in cell proliferation and cytokine production and to increased cell apoptosis in response to mitogenic stimuli, but not to abnormal thymopoiesis. J Immunol, 162, 6442-50.
Franzoso, G., Bours, V., Park, S., Tomita-Yamaguchi, M., Kelly, K. and Siebenlist, U. (1992) The candidate oncoprotein Bcl-3 is an antagonist of p50/NF-kappa B-mediated inhibition. Nature, 359, 339-42.
Franzoso, G., Carlson, L., Xing, L., Poljak, L., Shores, E. W., Brown, K. D., Leonardi, A., Tran, T., Boyce, B. F. and Siebenlist, U. (1997) Requirement for NF-kappaB in osteoclast and B-cell development. Genes Dev, 11, 3482-96.
Fujita, T., Nolan, G. P., Liou, H. C., Scott, M. L. and Baltimore, D. (1993) The candidate protooncogene bcl-3 encodes a transcriptional coactivator that activates through NF-kappa B p50 homodimers. Genes Dev, 7, 1354-63.
Fujita, T., Shibuya, H., Ohashi, T., Yamanishi, K. and Taniguchi, T. (1986) Regulation of human interleukin-2 gene: functional DNA sequences in the 5′ flanking region for the gene expression in activated T lymphocytes. Cell, 46, 401-5.
Gerondakis, S., Grossmann, M., Nakamura, Y., Pohl, T. and Grumont, R. (1999) Genetic approaches in mice to understand Rel/NF-kappaB and IkappaB function: transgenics and knockouts. Oncogene, 18, 6888-95.
Ghendler, Y., Teng, M.-K., Liu, J.-H., Witte, T., Liu, J., Kim, K., Kern, P., Chang, H.-C., Wang, J.-H. and Reinherz, E. (1998) Differential thymic selection outcomes stimulated by focal structural alteration in peptide/major histocompatibility complex ligands. Proc Natl Acad Sci USA, 95, 10061-10066.
Ghosh, S., May, M. J. and Kopp, E. B. (1998) NF-kappa B and Rel proteins: evolutionarily conserved mediators of immune responses. Annu Rev Immunol, 16, 225-60.
Goldrath, A. W. and Bevan, M. J. (1999) Selecting and maintaining a diverse T-cell repertoire. Nature, 402, 255-62.
Gong, Q., Cheng, A. M., Akk, A. M., Alberola-Ila, J., Gong, G., Pawson, T. and Chan, A. C. (2001) Disruption of T cell signaling networks and development by Grb2 haploid insufficiency. Nat Immunol, 2, 29-36.
Gorina, S. and Pavletich, N. P. (1996) Structure of the p53 tumor suppressor bound to the ankyrin and SH3 domains of 53BP2. Science, 274, 1001-5.
Gross, A., McDonnell, J. M. and Korsmeyer, S. J. (1999) BCL-2 family members and the mitochondria in apoptosis. Genes Dev, 13, 1899-911.
Haruta, H., Kato, A. and Todokoro, K. (2001) Isolation of a novel interleukin-1-inducible nuclear protein bearing ankyrin-repeat motifs. J Biol Chem, 276, 12485-8.
Hattori, N., Kawamoto, H., Fujimoto, S., Kuno, K. and Katsura, Y. (1996) Involvement of transcription factors TCF-1 and GATA-3 in the initiation of the earliest step of T cell development in the thymus. J Exp Med, 184, 1137-47.
Hettmann, T., DiDonato, J., Karin, M. and Leiden, J. M. (1999) An essential role for nuclear factor kappaB in promoting double positive thymocyte apoptosis. J Exp Med, 189, 145-58.
Horvitz, H. R., Ellis, H. M. and Stemberg, P. W. (1982) Programmed cell death in nematode development. Neurosci. Commentaries, 1, 56-65.
Huang, T. T., Kudo, N., Yoshida, M. and Miyamoto, S. (2000) A nuclear export signal in the Nterminal regulatory domain of IkappaBalpha controls cytoplasmic localization of inactive NF-kappaB/IkappaBalpha complexes. Proc Natl Acad Sci USA, 97, 1014-9.
Hubank, M. and Schatz, D. G. (1994) Identifying differences in mRNA expression by representational difference analysis of cDNA. Nucleic Acids Res, 22, 5640-5648.
Huxford, T., Huang, D. B., Malek, S. and Ghosh, G. (1998) The crystal structure of the IkappaBalpha/NF-kappaB complex reveals mechanisms of NF-kappaB inactivation. Cell, 95, 759-70.
Ivanov, V. and Ceredig, R. (1992) Transcription factors in mouse fetal thymus development. Int Immunol, 4, 729-37.
Jacobs, M. D. and Harrison, S. C. (1998) Structure of an IκBα/NF-κB complex. Cell, 95, 749-758.
Johnson, C., Van Antwerp, D. and Hope, T. J. (1999) An N-terminal nuclear export signal is required for the nucleocytoplasmic shuttling of IkappaBalpha. EMBO J, 18, 6682-93.
Jones, D. T. (1999) Protein secondary structure prediction based on position-specific scoring matrices. J Mol Biol, 292, 195-202.
Karin, M. and Ben-Neriah, Y. (2000) Phosphorylation meets ubiquitination: the control of NF-κB activity. Annu. Rev. Immunol., 18, 621-663.
Kerr, J. F., Wyllie, A. H. and Currie, A. R. (1972) Apoptosis: a basic biological phenomenon with wide-ranging implications in tissue kinetics. Br J Cancer, 26, 239-57.
Kitamura, H., Kanehira, K., Okita, K., Morimatsu, M. and Saito, M. (2000) MAIL, a novel nuclear IκB protein that potentiates LPS-induced IL-6 production. FEBS Letters, 485, 53-56.
Lee, S. L., Wesselschmidt, R. L., Linette, G. P., Kanagawa, O., Russell, J. H. and Milbrandt, J. (1995) Unimpaired thymic and peripheral T cell death in mice lacking the nuclear receptor NGFI-B (Nur77). Science, 269, 532-5.
Lerner, A., Clayton, L. K., Mizoguchi, E., Ghendler, Y., von Ewik, W., Koyasu, S., Bhan, A. K. and Reinherz, E. L. (1996) Cross-linking of T-cell receptors on double-positive thymocytes induces a cytokine-mediated stromal activation process linked to cell death. EMBO J, 15, 5876-5877.
Lisitsyn, N., Lisitsyn, N. and Wigler, M. (1993) Cloning the differences between two complex genomes. Science, 259, 946-951.
Liu, Z. G., Smith, S. W., McLaughlin, K. A., Schwartz, L. M. and Osborne, B. A. (1994) Apoptotic signals delivered through the T-cell receptor of a T-cell hybrid require the immediate early gene nur77. Nature, 367, 281-4.
Mandiyan, V., Andreev, J., Schlessinger, J. and Hubbard, S. R. (1999) Crystal structure of the ARF-GAP domain and ankyrin repeats of PYK2-associated protein beta. Embo J, 18, 6890-8.
McDonald, N. Q. and Peters, G. (1998) Ankyrin for clues about the function of p161NK4a. Nat Struct Biol, 5, 85-8.
Meuer, S. C., Hussey, R. E., Cantrell, D. A., Hodgdon, J. C., Schlossman, S. F., Smith, K. A. and Reinherz, E. L. (1984) Triggering of the T3-Ti antigen receptor complex results in clonal T cell proliferation through an interleukin 2-dependent autocrine pathway. Proc Natl Acad Sci USA, 81, 1509-1513.
Penninger, J. M., Sirard, C., Mittrucker, H. W., Chidgey, A., Kozieradzki, I., Nghiem, M., Hakem, A., Kimura, T., Timms, E., Boyd, R., Taniguchi, T., Matsuyama, T. and Mak, T. W. (1997) The interferon regulatory transcription factor IRF-1 controls positive and negative selection of CD8+ thymocytes. Immunity, 7, 243-54.
Plaisance, S., Vanden Berghe, W., Boone, E., Fiers, W. and Haegeman, G. (1997) Recombination signal sequence binding protein Jkappa is constitutively bound to the NF-kappaB site of the interleukin-6 promoter and acts as a negative regulatory factor. Mol Cell Biol, 17, 3733-43.
Rathmell, J. C. and Thompson, C. B. (1999) The central effectors of cell death in the immune system. Annu Rev Immunol, 17, 781-828.
Reinherz, E. L., Tan, K., Tang, L., Kern, P., Liu, J., Xiong, Y., Hussey, R. E., Smolyar, A., Hare, B., Zhang, R., Joachimiak, A., Chang, H. C., Wagner, G. and Wang, J. (1999) The crystal structure of a T cell receptor in complex with peptide and MHC class II. Science, 286, 1913-21.
Saitou, N. and Nei, M. (1987) The neighbor-joining method: a new method for reconstructing phylogenetic trees. Mol Biol Evol, 4, 406-25.
Sasada, T., Ghendler, Y., Wang, J. H. and Reinherz, E. L. (2000) Thymic selection is influenced by subtle structural variation involving the p4 residue of an MHC class I-bound peptide. Eur J Immunol, 30, 1281-9.
Schreiber, E., Matthias, P., Muller, M. M. and Schaffner, W. (1989) Rapid detection of octamer binding proteins with ‘mini-extracts’, prepared from a small number of cells. Nucleic Acids Res, 17, 6419.
Sebzda, E., Mariathasan, S., Ohteki, T., Jones, R., Bachmann, M. F. and Ohashi, P. S. (1999) Selection of the T cell repertoire. Annu Rev Immunol, 17, 829-74.
Seder, R., Paul, W., Davis, M. and Fazekas de St. Groth, B. (1992) The presence of interleukin 4 during in vitro priming determines the lymphokine-producing potential of CD4+ T cells from T cell receptor transgenic mice. J Exp Med, 176, 1091-1098.
Sellins, K. S. and Cohen, J. J. (1987) Gene induction by gamma-irradiation leads to DNA fragmentation in lymphocytes. J Immunol, 139, 3199-3206.
Senftleben, U., Li, Z., Baud, V. and Karin, M. (2001) IKKβ is essential for protecting T cells from TNFα-induced apoptosis. Immunity, 14, 217-30.
Sloan-Lancaster, J., Shaw, A. S., Rothbard, J. B. and Allen, P. M. (1994) Partial T cell signaling: altered phospho-zeta and lack of zap70 recruitment in APL-induced T cell anergy. Cell, 79, 913-22.
Smith, C. A., Williams, G. T., Kingston, R., Jenkinson, E. J. and Owen, J. J. (1989) Antibodies to CD3/T-cell receptor complex induce death by apoptosis in immature T cells in thymic cultures. Nature, 337, 181-184.
Steller, H. (1995) Mechanisms and genes of cellular suicide. Science, 267, 1445-1449.
Tam, W. F. and Sen, R. (2001) Ikappa B Family Members Function by Different Mechanisms. J Biol Chem, 276, 7701-7704.
Thompson, J. D., Gibson, T. J., Plewniak, F., Jeanmougin, F. and Higgins, D. G. (1997) The CLUSTAL_X windows interface: flexible strategies for multiple sequence alignment aided by quality analysis tools. Nucleic Acids Res, 25, 4876-82.
Turner, J. M., Brodsky, M. H., Irving, B. A., Levin, S. D., Perlmutter, R. M. and Littman, D. R. (1990) Interaction of the unique N-terminal region of tyrosine kinase p56lck with cytoplasmic domains of CD4 and CD8 is mediated by cysteine motifs. Cell, 60, 755-65.
Vaux, D. L. and Korsmeyer, S. J. (1999) Cell death in development. Cell, 96, 245-54.
Verma, I. M., Stevenson, J. K., Schwarz, E. M., Van Antwerp, D. and Miyamoto, S. (1995) Rel/NF-kappa B/I kappa B family: intimate tales of association and dissociation. Genes Dev, 9, 2723-35.
Voll, R. E., Jimi, E., Phillips, R. J., Barber, D. F., Rincon, M., Hayday, A. C., Flavell, R. A. and Ghosh, S. (2000) NF-κB activation by the pre-T cell receptor serves as a selective survival signal in T lymphocyte development. Immunity, 13, 677-689.
von Boehmer, H. (1991) Positive and negative selection of the alpha beta T-cell repertoire in vivo. Curr Opin Immunol, 3, 210-215.
Weih, F., Durham, S. K., Barton, D. S., Sha, W. C., Baltimore, D. and Bravo, R. (1997) p50-NFkappaB complexes partially compensate for the absence of RelB: severely increased pathology in p50(−/−)relB(−/−) double-knockout mice. J Exp Med, 185, 1359-70.
Wyllie, A. H. (1980) Glucocorticoid induced thymocyte apoptosis is associated with endogenous endonuclease activation. Nature, 284, 555-556.
Yamazaki, S., Muta, T. and Takeshige, K. (2001) A novel I{kappa}B protein, I{kappa}B-{zeta}, induced by proinflammatory stimuli, negatively regulates nuclear factor-{kappa}B in the nuclei. J Biol Chem, 16, 16.
Zhang, B. and Peng, Z. (2000) A minimum folding unit in the ankyrin repeat protein p16(INK4). J Mol Biol, 299, 1121-32.

The contents of all references, patents and published patent applications cited throughout this application are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. An isolated nucleic acid molecule consisting of a nucleic acid sequence selected from the group consisting of:
a) SEQ ID NO: 1;
b) the complement of SEQ ID NO: 1;
c) SEQ ID NO: 3;
d) the complement of SEQ ID NO: 3;
e) a portion of SEQ ID NO: 1 which is at least 500 nucleotides in length;
f) a portion of the complement of SEQ ID NO: 1 which is at least 500 nucleotides in length;
g) a portion of SEQ ID NO: 3 which is at least 35 nucleotides in length;
h) a portion of the complement of SEQ ID NO: 3 which is at least 35 nucleotides in length;
i) a nucleic acid sequence that encodes SEQ ID NO: 2; and
j) a nucleic acid sequence that encodes SEQ ID NO: 4.

2. An isolated nucleic acid molecule according to claim 1 which is DNA.

3. An isolated nucleic acid molecule according to claim 1 which is RNA.

4. An isolated nucleic acid molecule comprising a nucleic acid sequence selected from the group consisting of:
a) SEQ ID NO: 3;
b) the complement of SEQ ID NO: 3;
c) a portion of SEQ ID NO: 3 which is at least 35 nucleotides in length;
d) a portion of the complement of SEQ ID NO: 3 which is at least 35 nucleotides in length;
e) a nucleic acid sequence that encodes SEQ ID NO: 2; and
f) a nucleic acid sequence that encodes SEQ ID NO: 4.

5. An isolated nucleic acid molecule according to claim 4 which is DNA.

6. An isolated nucleic acid molecule according to claim 4 which is RNA.

7. An isolated nucleic acid molecule comprising a nucleic acid sequence that hybridizes under high stringency to a nucleic acid sequence selected from the group consisting of:
a) SEQ ID NO: 1;
b) the complement of SEQ ID NO: 1;
c) SEQ ID NO: 3;
d) the complement of SEQ ID NO: 3;
e) a portion of SEQ ID NO: 1 which is at least 500 nucleotides in length;
f) a portion of the complement of SEQ ID NO: 1 which is at least 500 nucleotides in length;
g) a portion of SEQ ID NO: 3 which is at least 35 nucleotides in length;
h) a portion of the complement of SEQ ID NO: 3 which is at least 35 nucleotides in length;
i) a nucleic acid sequence that encodes SEQ ID NO: 2; and
j) a nucleic acid sequence that encodes SEQ ID NO: 4.

8. A probe comprising a nucleic acid sequence that hybridizes under high stringency conditions to a nucleic acid sequence selected from the group consisting of:
a) SEQ ID NO: 1;
b) the complement of SEQ ID NO: 1;
c) SEQ ID NO:3;
d) the complement of SEQ ID NO: 3;
e) a portion of SEQ ID NO: 1 which is at least 500 nucleotides in length;
f) a portion of the complement of SEQ ID NO: 1 which is at least 500 nucleotides in length;
g) a portion of SEQ ID NO: 3 which is at least 35 nucleotides in length;
h) a portion of the complement of SEQ ID NO: 3 which is at least 35 nucleotides in length;
i) a nucleic acid sequence that encodes SEQ ID NO: 2; and
j) a nucleic acid sequence that encodes SEQ ID NO: 4.

9. An isolated polypeptide, or functional portion thereof, comprising an amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.

10. A vector or plasmid comprising a nucleic acid sequence selected from the group consisting of:
a) SEQ ID NO: 1;
b) the complement of SEQ ID NO: 1;
c) SEQ ID NO: 3;
d) the complement of SEQ ID NO: 3;
e) a portion of SEQ ID NO: 1 which is at least 500 nucleotides in length;
f) a portion of the complement of SEQ ID NO: 1 which is at least 500 nucleotides in length;
g) a portion of SEQ ID NO: 3 which is at least 35 nucleotides in length;
h) a portion of the complement of SEQ ID NO: 3 which is at least 35 nucleotides in length;
i) a nucleic acid sequence that encodes SEQ ID NO: 2; and
j) a nucleic acid sequence that encodes SEQ ID NO: 4.

11. The vector or plasmid of claim 10 wherein the isolated nucleic acid sequence is operatively linked to a regulatory sequence.

12. A recombinant host cell comprising the vector or plasmid of claim 11.

13. A method for preparing a polypeptide encoded by an isolated nucleic acid sequence, comprising culturing the recombinant host cell of claim 12.

14. A fusion protein comprising the amino acid sequence of an isolated polypeptide of selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3;
or functional fragment thereof.

15. An antibody or antibody fragment that binds to a portion of a polypeptide molecule having an amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.

16. The antibody or antibody fragment of claim 15 which is polyclonal.

17. The antibody or antibody fragment of claim 15 which is monoclonal.

18. A method for determining the presence or absence of a polypeptide comprising an amino acid sequence of SEQ ID NO: 2, or portion thereof, in a sample comprising:
a) obtaining the sample to be tested;
b) contacting said sample with an antibody specific to a polypeptide molecule having an amino acid sequence of SEQ ID NO: 2, or a portion thereof, to allow formation of a complex between the polypeptide and the antibody; and
c) detecting the presence or absence of complex formation.

19. A method for determining the presence or absence of a polypeptide comprising an amino acid sequence of SEQ ID NO: 4, or portion thereof, in a sample comprising:
a) obtaining the sample to be tested;
b) contacting said sample with an antibody specific to a polypeptide molecule having an amino acid sequence of SEQ ID NO: 4, or a portion thereof, to allow formation of a complex between the polypeptide and the antibody; and
c) detecting the presence or absence of complex formation.

20. A method to identify protein-interaction partners that interact with an isolated polypeptide, or functional portion thereof, comprising an amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.
comprising contacting said polypeptide with the agent to be tested and assaying for presence or absence of complex formation between the polypeptide and agent.

21. A method to identify an agent that alters the activity of an isolated polypeptide, or functional portion thereof, comprising an amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.
comprising contacting said polypeptide, or functional fragment thereof, with an agent to be tested, determining the level of activity of the polypeptide in the presence of the agent, and comparing said level of activity with the level of activity of the polypeptide, in the absence of the agent, wherein a statistically-significant change in activity is indicative that the agent alters the activity of the polypeptide.

22. The method according to claim 21 wherein the activity is altered NFκB-induced expression.

23. The method according to claim 21 wherein the agent identified is an agonist.

24. The method according to claim 21 wherein the agent identified is an antagonist.

25. A non-human transgenic animal comprising a recombinant nucleic acid molecule comprising a nucleic acid sequence selected from the group consisting of:
a) SEQ ID NO: 1;
b) the complement of SEQ ID NO: 1;
c) SEQ ID NO: 3;
d) the complement of SEQ ID NO: 3;
e) a nucleic acid sequence that encodes SEQ ID NO: 2; and
f) a nucleic acid sequence that encodes SEQ ID NO: 4.

26. A non-human transgenic animal comprising a recombinant nucleic acid molecule encoding a polypeptide molecule having an amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.

27. A method to identify an agent that alters the activity of a polypeptide molecule having an amino acid sequence selected from the group consisting of:
a) SEQ ID NO: 2;
b) SEQ ID NO:4;
c) an amino acid sequence encoded by SEQ ID NO: 1; and
d) an amino acid sequence encoded by SEQ ID NO: 3;
comprising exposing a transgenic animal of claim 25 to the agent to be tested and determining the level of activity of the polypeptide or functional fragment thereof, wherein increased activity indicates that the agent is an agonist, whereas decreased activity indicates that the agent is an antagonist.

28. The method according to claim 27 wherein the activity is altered NFκB-induced expression.

29. A non-human transgenic animal comprising the deleted nucleic acid sequence of SEQ ID NO: 3, or fragment thereof.

30. A method to identify gene targets of a polypeptide comprising, determining the gene expression in a non-human transgenic animal comprising an isolated nucleic acid molecule encoding said polypeptide molecule having an amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.
and comparing the gene expression with a non-human transgenic animal comprising a deleted nucleic acid sequence of SEQ ID NO: 4, or fragment thereof, wherein genes identified to be expressed in association with expression of said polypeptide, are potential targets of the polypeptide.

31. A method to identify gene targets of a polypeptide with amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.
comprising determining the gene expression in a recombinant host cell containing a polypeptide with said amino acid sequence, and comparing the gene expression with a non-recombinant host cell, wherein genes identified to be expressed in association with expression of the polypeptide, are potential targets of the polypeptide.

32. A method of treating an individual having a disorder by administering a therapeutically-effective amount of an agonist of a polypeptide with amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.

33. The method according to claim 32 wherein the disorder is an autoimmune disease.

34. The method according to claim 33 wherein the autoimmune disease is selected from the group consisting of, rheumatoid arthritis, diabetes mellitus and multiple sclerosis.

35. The method according to claim 32 wherein the agent is administered orally, intravenously, intramuscularly, subcutaneously, topically, rectally, or by inhalation.

36. A method of treating an individual having a disorder by administering a therapeutically-effective amount of an antagonist of a polypeptide with amino acid sequence selected from the group consisting of:
a) SEQ ID NO: 2;
b) SEQ ID NO: 4;
c) an amino acid sequence encoded by SEQ ID NO: 1; and
d) an amino acid sequence encoded by SEQ ID NO: 3.

37. The method according to claim 36 wherein the disorder is selected from the group consisting of cancer, malaria, tuberculosis, and HIV infection.

38. The method according to claim 36 wherein the agent is administered orally, intravenously, intramuscularly, subcutaneously, topically, rectally, or by inhalation.


SEQUENCE LISTING (12 sequences)

SEQ ID NO: 1 - DNA, 15000 bp, Homo sapiens
cagtagcagtctgatttcctaggttctcttgagtccagtagtctatgcacatagtcctacggagccccagggcccaggctcagcctttttagtgccccaggagtccaggccctcaattcctcagtgtcccaggaatccaggtcccgggagcttggtgtcccagaagtactaactctcagtaccttgggagacgccaggccctctagccctggatgtgacgtccccaactctggtaaccagacctgcccctgcccctcctcttacacgcaggccaccctcccttcccagcacactcggagactgtgggttctggacctagcagcctgggctttccagactgggaccccaacacgcatgctgcctacactgacagcccctactcttgccctgcttctgctgccgaaaatttcctgcctcctgacttctacccaccctcggacccagggcagccgtgcccatttccccagggcatggaggtgagatacagagtgggtacaggatgctgaagggcccctaaaaattcgcagttgcactcctgcagtattctgagatgtctggagctcagttacgattcccttttccaggccgggcgtggtggctcacacctgtaatcccagcactttgggaggctgaggcaggcggatcacttgaggtcaggcgtttgagaccagcttggccaacatggtgaaaccaaaaataaaaaaattagctgggtgtagtggcgcacgcctgtaatcctagctactcgggaggctgaggcaggggaattgcttgaacccaggaggtagagattgcagtgagtagagattgcactgctgcactccagcctgggtgacagagcaagactctgtcaccgaaaaaaaaaaatacccttttccaggtgggcaaaactgaggcctaaaggtccaaggtcacttggcatagatgggatacccaggggctctgcatgcagatgtgtgtgtggtggcttgggatggaggaattctcagctgactgggtctccccaccacctgcaccctccaggaccccccagacacccagttctatgtaggatcttctctgccacaggctggaccctggagagtttctgcacccccttcaggacccccacagttccccgctgtggtccctggaccatcgctggaggtggcccgagctcacatgctggctttggggccacagcagctgctggcccaggatgaggagggggacacgtgagtataagggatagggttgtctgcagactcttggcttggtgggggctgttctcacggctgtcccccacctgtcctcaggctccttcacctgtttgcggctcgggggctgcgctgggcggcatatgctgcggctgaggtgctccaggtgtaccggcgtcttgacattcgtgagcataagggcaaggtgaggcttggggcctgaactctcctgggttcaggggaggaaggagcctggggcctggacccctggatcctagagaagaggggaatggggggtgagctcctggggctgaggggggatggggctgggagcctggttttctgacgggagggaaccaggagcccaacctgacttctcctgtggcctgcagacccctctcctggtggcggctgctgccaaccagcccctgattgtggaggatctgttgaacctgggagcagagcccaatgccgctgaccatcagggacgttcggtcttgcacgtggccgctacctacgggctcccaggagttctcttggtatggccagctggcaggcaggggtttgtctggggggtagactggttgcccagattttggcttccagggccaggaggccaggggatacccttacccagcagtctgccttctcttcctcccaggctgtgcttaactctggggtccaggttgacctggaagccagagacttcgagggtaagttgggtgtgggcagggctggtgtggttggtgtggctgggcgaggtgggtgcagatgcgggtccctcactgtctccattctgcaggcctcaccccgctccacacggccatcctggcccttaacgttgctatgcgcccttccgacctctgtccccgggtgctgagcacacaggcccgagacaggctggattgtgtccacatgttgctgcaaatgggtgctaatcacaccagccaggtgagcggggatgtgggcggtcagaccctgggagattgtgtggaatggggccacttggatgtccgggagctccaggcaaatgctgactttgcctcttccttgctgcgtgcccttgaacatgttacttcacttctctgtgccttaggggttttttgtttgtttctttgtttgttttgagatggagttttgctcttgttgccctggctggagtgcaatggtgtgatcttggctcaccgcaacctctgcatcccgggttcaagtgattctcctgcctcagcctcctgagtagctgggattacaggcatgagtcaccacgcctggctaatttttttgtatttttagtagagacaggctttctccacattggtcttgctggtctcgaactcccgacctcaggtgatccacctgccttggcctcccaaagtgctgtgattacaggcgtgagccaccgcgcccagcagggtttttttgtttgtttgtttttttgtttttgagacagggtctcactctgtcgcccaggctagagtgcagtggtgcaatctcatctcatggcaaactctgcctcccaggttcaagtgattctcctgtcttagcctcctgagtagctgggactataggcgcacgccaccatgcctggctatttttttttttttttttttgtattttagtagaagtgggctttcattgtgttggccaggctggtctcaaactcctgacctcaaatgatcctcccaccttggcctcccaaagtgctgcgattacaggcgtgagccactgtgcctggcccctagttgttaactgggaataatattactgaccttagggtatgttatcaagcacttactattcattcatcaagcactgagtttcatagattagcttattgaaacctcacaaacaccctttgacatgtgtactctattttctctattttactgaaaaggaaacaggcatgggaaatcaaaaatctttttttttttctttttgagatggagttttgctcttgttgcccaggctggagtgcaatggtgtgatctcggctcaccacaacctccgcctcctgggttcaagcgattctcctgcctcagcctcccgagtaactgggattacaggttcccaccaccacgtccagctaattttgtatttttagtagagacagggtttctccatgttggtcaggctggtctcgaactcccgacctcaggtgatcttcctgcctcggcttcccaaaatgctgggattgcaagcgtgagccactgcacctggcctcaaaaatctctttgagacagggtctcactctgtcactcaggctggagtgcagtggcatgatcacagcttattgcagccttgaccttgtgcacttaagtgatcctcccacctcagcctcccaagtagctgtgatcacaggcatgcaccaccatgcgtggcaacttttttgtaaggtggggtctaactatgttggccaggttggtctcaaactcctgagctcaagcaatccttccacctctgcctctgaaagtgctgggattacagttgtgagccactgcacccagcctgaaaaatcttatagcttgtgactgtcagagagagaattagaacccaggtagtctgactttagaacctagcttttaattatgttctgttcctttcctgtgacagtagctccttcataggctggctgagagaattaaacatccacaaaaaatgtttatttatttattgagatggaatctcactctgtcacccaggctggagtgcagtggcatgatctcggcttactgcaacctctgcctcccgggttcaagccatgctcctgcctcagcctcctgagtaactgggactacaggcatgcaccaccacgcttggctaatttttttgtatttttaatggagacggggtttccctatgttggccaggctgttctctcaaactcctgacctcaggtgagccgcccgccttggcctcccaaagtgctgggattacaggcataagccactgcgcccagccgaaaaatgtttttttagttaaagaatgttgaacaccaataaaatatagaaaatagtggaatgaactccatatgtccaacatcaataaccagccactcatggccagtcctgccccatccactctctcactttcaagtgattttttttttttttttgagatggctctcgctctgtcacccaggctggagtgcagtggtgcgatctcggctcactgtaagctccgcctcccgggttcacaccattctcctgcctcagcctcctgagtagctgggactataggcacccgccaccacgcccggctaaatttttgcatttttagtagagacgcagtttcaccgtgttagccaggatggtctcgatctcctgacctcgtgatccacccgcctcggcctcccaaagtgctgggattacaggcgtgagccactgtgcgtggcctctcactttcatgtgatttttaagcaaattccaacatgtatttttttcaaaaatatttgtgtatctttaaagaataatggtgtttagcagaattagctagatggtggggatggactgtaagaaggacgggcatgactgagtagagacaagataaggcattattagcggggttaggaaatgaatgatggtaatctaggctcttaatggttgcaaagtagagctaagtgatcatttgggcaatacagagaaggtgaaattgatactttggtaattcattcgtttcttcactcagaattttggacacatatgatgtagctaccagtgtgctagtgatggggcataaagttgctttttgtttgtttgttttttagagacagagtctggctttgtcacccaggctggagtgctgtggtgcgatcacgaatcactgcagcctcgacttcctgacctcaaacattccttccacctcagcctcctaagtagctgggactacaggcatgagccacctcacccggctaattgttttatattttttgtagagagggggggtctcactatgttgcccaggctggtctcgaactcctgggcccaagtgatcccccagccttggcctcccaagtgctgggattacaggcgtgagctgctgtgcccagctacctataatgttttaataagaaaagaccactttcttgtacttttagacattaaaatttaatggaaaagagaggagatgataagtaaacaaagaactgaacataattacaaattgggatgagagctgccttgggaatgtagaaggagctcttagacaataaaaagattggctttatattggatgcggggctcacaatgaaggtcattctgaggaaattacatttaagttgagcaagaaatgatgaagaggaagaattaactgtgtggtagggttctgggagaactcagaaatcagggagtatttctctgaaaaaaggacaaataattttttttttttttttgagatggagtctcgctctgtcgcccaggttggagtgcagtggcacagtcttggttcactgcaacctcccaggttcaagtgattctcctgcctcagcctcccgagtagctgggactataggcatgtgctatcacaccccactaatttttgtatttttagtaataataataagttttttaaaaataagaaagatggctgggtgcggtggctcatgcctgtaatccaagcactttgggaggctcaggtgggcggactgctcgagctcaggaattcgagaccaacctgggcaacatagggagaccccatctctaaaaaaaatacaaaaattagctgggcttgatggcacatgcctgtggtcccaggtactcaggaggctgaggtgggaggatcacctgaacctggggaggtcaaggctgcagtgagccatgattttgccaccacactccagcctgggtgacagagcaagaccctgtctctcaacaacaacaacaacaataataaaagataagaactctttccctgtaatcccagtgcttggggaggctaaggtgggaggatcactcgagcccaataggttgaggctacagtgagccttggtcacaccactgcactccagcctgggcaacagagcgagaccctgtctcaaaaaacaaacaaaaaacatagctttttttttttgagacggagtctcattctgttgcccaggctggattgcaatggtgtgattttggctcactgcaacctctgcctcccgggttcaagctattcctgtgtctcagcctcccaagtagctgggattataggcatgtgccaccacgcctggctaatttttgtatttttagtagagacggtttcaccatgttggccaggctggtctcgaactcttgacctcaagtgatccgccctccttggcctcccaaagtgctgggattacaggcatgagcccctgtgcccttcgaaaaaaaagaactttttaaacctcagtatcattgctctgtctttaaaaaattaaagattttcttgtaataccaaatatcctggctgtattcaaatttccttttggctgtaaaatgataatttttttcattaaaaaaaatcaagatccaaataagcacacatattatgatgttacgtcttttcaaatcactcttaatctagagctttggttttgtcttcatcattttttggacgagcaaactaggtggttttgccctgttatcagcacataaaatgtttaacattcattatcgttacaaactccttaaagacacataaaaaggtttcattttagaaaatgtcttttattttcccccaaatatgatgggaacagctcaatattgtggtgcagtattgcaatattagctagtccactgattaatgctgccaactcctgtgcttgtagataaagtacagtctgtgtgggtcgggatgcaatttaatgtgttgaatggagggcaggtagccaaggcagttgctttgaagaactggaaaaggagtctctttctcagacactggtgggaaaattgtcatgagaaatgtacagttctggaaggtgtatggtgtgaatggtacaccctgagacgtggtgttgtgcggtgcacaagacatacaactgaataaggcactcttggtggggctcgcagaagagggccagcatcttatttatttatttattaattttgagatagagtgtcactctgtcacccatgctggagtgcagtggtgtggtcatggcccactgcagccgtgaactcctaggctcaaatgaacctcctgcctcagcctcccaagcagctgggactacaggtgcgcaccaccacgcctggctaatttttctattttttgtagagatgaggtcttgctgtgttgtccaggctggtctgcagctcctggcctcaagtgatcctccttcttcagcttcccaaagtgctaggattacaggcgtgaaccaccgcactccgccaagggtcagggtctttaaagactcctggattctgtgggagggcacctaggtgtgaggctggccctgacctctgccattcccttcccctcaggagatcaagagcaacaagacagttctgcacttggccgtgcaggctgccaaccccactctggttcagctgctgctggagctgccccggggagacctgcggacctttgtcaacatgaaggtgggagctggggctgggaagtgcagattgtgtggggtacggtagggggggcagtgggatgttaggggtcctgggggctgtggacgtggaggggacgatgtgtgggatctgcaggggaaagtataggaagcagacacagattgccccagaacatgtagtccgtggacacagggatgcatgcagcgtggctgtggccgggggaggtctgggatgtgaaaggacaagtgtaggacgtagacaaggcagaaattgaagtgcagtttagaggcctgggccagctgagcccagatccccaccagccctccccttacttttccaggcccacgggaacacagccctccacatggcggctgccctgccccctgggccggcccaggaggccatcgtgcggcacctgttggcagctggggcggaccccacactgcgcaacctggagaatgagcagcccgttcacctgctgcggcccgggccgggccctgagggggtaagtaagcactgagcctgcctgtagagtggcctctgagctcgcgggcgcgggatgacctccctgccccacccaatgctcgctttcagagaatgacttctaatctcggaaggtgacttcagccctcaggcaaccctgactctataatgtgacctctgacctcagtgtttcatcctttaccccagaatggtgacttctctgatctctaccccaaccctgacctcacagtgtgacctgtacttccaaccctgaatcttgcttgacctcaaggtgtgacctttgatccccccaagtgtgacctcctaccccctgcccctaaccttgggtcctgacctcagatgtgacctttgacctcttacctgttcccttcaattccctctcattcctcacccccaggtgggaacccccagccagcagcccctttcccttcactgaagttctgcttcctgaccctttctcagtgtacgaacccaggccttgaccttcttcccacatctttatttttatttgtttgtttgtttatttatttattttgaggcagcgtcttgctctgttgcctaggctggagtgcagtggcatgatcatagctcactgcagcctcgagctcctgggctcaagggatcctcccacctcagcctctccagtagctggtactacaggcagtcaccaccacacccagctaattttgtttttttttttttttaagagatggggtcacactatgttgtccaggttgatctcaaactcctggcctcaattgatcctcccgccttggcctcccaaagtgttgggatcacaggaaccatcactcccggcctccttcccaaatcttttttttttttttggaggcggagtcttgctttgtcacccaggctggagtgcagtggtgcaattttagctcaccgcaagctccacctcccaggttcaagtgattctcccacctcagcctcccaagctgggactaaaggcacacgtgccaccatgcccggctaatttttgtatctctggtagaggcggggtttcgccatgttggctgggctggtcttgaactcctgacctcaggtgatttgcccaccttagccccacaaagtgctgggattacaggcgtgagccactgcacctggctcttccaaaatcttgactctggacctctgatcttcaaatccaaagggacccactcaacctctcttctctagcccctccctctgcatggcgtgccttccccacctctccttccaacctgacagacttctctccctcctttgccttgtctacagctccggcagctgttgaagaggagccgtgtggcgccgccaggcctgtcctcttaggactcaaacccagaccctggactgattttccagtccccaccgtcctgcgggacagccagcgtatgctaatgttgcaaacccatgataatgtatgtggaatatcctgccattggggttttacattaaaaccccagaatggctgcagaggggtgaacaggccccaatatttggggtgctgtgatacccctcttctacccacaaggagccctcttgatgatttctgtgaaatcgaggccccttgattgtttctgtgaaacaccctgcacccctagtcctttccccactgagatctttcgggttctctcccctaactcagctcttcgttcccagaaacccagatgtaatccccctacgtggtgcttggggcatcccgataccatctcagtaaatctcctacaatggcctcctcaccctccccgggacccacacccttcaggtcctcaccctgagacaggagggaccctctgagatcagggacccttaggtctcactgctctctgattcagagctcagctgggcccccagttccagaccccagcattcccggtcactccctccctaatctgagcatcactcaagctctttattaaactcaatttgggccagatttggtggctcatgcctgtaatcctaacactctgggaggccgaggcgggcggatcacttgaggtgaggagtttgagaccagcctggccaacatggtgaaaccctgtctccactaaaaatacaaaaacaattggctggacatggtggcatgcgcctgtagtcccagctacttgggaggctgaggcagaagaattgattgaacccaggaggcggaggttgcagtgagccgagattgtgccactgcactccagcctaggtgacaaagagagactctgtctcaaaaaaaaaaaaaaaaaaaaatttaaactcaatttggactagaaccactgttgtctgatctctgggggcactggtgcaggggagtggctgtgatgcccgctgaggggtcatattgggatgaatgtgcctgaggggggaggatgtagcagccaccatgggtgccaagtccatatcctgtcaccctcaccctggcgacacaagagggactggcctttaggaggccaaggtgtgaggatcacttgaggccaggagtttgagaccagcctgggtgtcagccccagaatatttgcctttgggcaatctcaaaccactggtgcggctctgccccaggtatgagcatggcaagtgttgaaagccagggggttcgtgtccacttcccactccagcagccctcagccagttactgataggagatagtcccccacctgtctgttgggctaacttggaagtgcatggcctatgcgggctccagagttccccagcagatgaatccccagttccccacagtggtctgggcttgataaaataccctttattggctgctctttcttctctgtgtctcttcctgtctcccctaccaatgttctccatcccttccaaatgaactttccctccaatccttccctccatagctgcttttggagaaacccaaactaagaattccacaaaaccccattctcagccaggcgcagtgactcacacttaaaattgcagcactttgtgaggccgaggcgagcagatcacctgaggtcaggagtttgagaccagcctggccaacatggcaaaaacccatctctactaaaaatacaaaaattagccaggcgtggtggcgtgcgcctgtaatcccagctactcagaaggctgaagcatgagactcgcttgaacctgggaggcagaggttgcagtgagctgagattgcgccactgcactccaacctgggcgacagagcaagactctgtctcaaaaaaagatacaaaaaaacccccaaggctgggcgcagtggcttatgcctataatccctgcactttgggaggcggaggcgggtggactacctgaggtcgggagtttgagaccagcctggccaacatgatgaaaccttgtctctactaaaaatacaaaaaaattggccaggcgcagtgactcacacctgtaatcccagcattttgggaggccgaggcgggcggatcatgcggtcaggagattgagaccatcctggctaacacggtgaaaccctatctctactaaaaatacaaaccaaaaaaaaaaaaaattagctaggcatggtggcgggcacctgttttcccacctacttgggaggctaaagcaggaaaattccttaaatccaggaggcggaacttccattaacctaaaatcccaccacttttctccaccctgggcaacaaaacaaaattccgtctcaaaaaaaaaaaaaaacccaaaccccattctcatacccgctccacagcctctgctctggcctctgcaatataatcacacaaagagcccccatcccagaaagcccttggacccatcacagcccccatagctaggaacctttcatggaccacagaccgaatgtacatgttagcacattccggggctccagccaatgtctccaccccaaaacttaaggcttaaaccaacacccttttaattatttcttttcttttttcttttttgagaaggagtctcagtctcaccagattggagtgcagtggcgcgatctctgctcactgcaacctccgccttccaggttcaagcgactctcctgcctctgcctcccaagtagctgggattacaggcacccaccaccacgtctagctaatttttgtatttgtttagtagagattggggttccagcatgttggccaggcttgtctcgaactcctgacctcaagtgatccacccacctcggcctaccaaagtgctgggattacaggtgtgagctaccgcgcccagcccctcttaattgtttctgactgaaatctacgccgggttcagctggctgttctgctgtctccctttggttcctcatgtggctgcaatcaacctgaggcccaactggcctggaaatccacaacaccttcactcacatgcctgatagctcagctgagataatggagcttccaggagtggcccaagagtctttccatgcagtccaggagggcaggtggatctcttaacttcaggtggtggctcagtggttccaagaaagagagatccaaggagacaagtccctgcttgtatcatacttgttgacatgtcattgatcccagatcagtgtgggaggcattacacaagggtctcaatattgggagacagagttatctggggactgggcgaggtgcctcactcctgtaatcccagcaatttgggaggccaaggagggcggattacttgaggtcaggagttggagaccagcctggtcaacatggtgaaaccctgtctctactaaaaatacaaaaattagctgggcatggtggcgggtgcctgtagtcccagctactttggaggctgaggcaggaggatcacttgaggccaggagtttgatgccgcagtgaaccatgattgcagcactgcactccagcgtgggaggcagcaagacactgtctctaaagtaagtaaataaataataataataaaataaaaaatacagaagaacttaattgggatcatcttatagtcaattgtgtgacatagtttatattttgccttttttttccttttgtggtacctaaaataatgctattttttatcactgatggcatctgaggttttgttgacatataagatgtatatctcaatttgtattgttatggagtgcatgtttgtgtccctctaaaattcatttgttgaaaccataagccccagtggggtagtatttgaagatgggcctttgggaggtagttaggtcatgagggtggagccctcatgaatgggactaatgtccttgttagaagagatgggagagtccgggcgtggtggctcatgcctgtaatcccagcactttgggaggccgaggcgggtggatcatctgaggtctggagttcgagaccagcctgaccaacatggagaaaccccgtctctactaaaaaatacatattagccaggcatggtggtgcatgcctgtaatcccagctactcaggaggctgaggcaggagaatcacttgaacctgggaggtggagattgcggtgacccgagatcatgccattgcactccatcctgggcaacaagagtgaaactccatctcaaaaaaaaaaaaaaaagaaggagacaggagagagcttgcttctctgctctctactctctgccatatgaagatgcagtaagaggatggccatcttgggcagggcacactggctcacacctgtaatcccagcattttgggaggccaggatgggaggatagcttgagctcaggagtttgagaacagcgtgggcagcatggtgaaatcctgtctctactcaaaaaaaacaaaatagccaggtgtagtggtgcacacctgtggtcccagctactcgagagagtaaggcaggagaattgcctcagcctgggagatcaaggctgcagtgagctgtgattgcaccagtgcactccagccaggtgacagaacaagaccttgtcaaaaaacaaaacaaaacaaaccatctgcaaaccaggaagagagtcctccagactttggagctgctggcaccttgatcttggacttctcagcctctagcacttgtgagaaataaatgtgtgttgtttaagccacccagctcatggtattcttagcccagactaagatgcatatcaatacacacatgtgcacacacccaccccactaacatgataaaatgtccctcagaaactttgtgtgtttttttttttttttttttttaaataagaacagggtctcactcttgctcaggctggagtacagtggtgcaatgtcagctcactgcagcctccacctcctgcctggactcaagagatccttctgcctcagcctcttgagaagctaggaccacaggtgtatgccaccacacccagctaattttttcgtttctatttttgtagatacagggtctcactatgttgcccaggctggtctcgaactcctgggctcaagggatactcccacctcggcctcccaaagtgctgggattacatgcatgagcttctgcacctgtccatatattattattattattattattttgagaccgggtctcaatctgtcttctttggctctctacagcctcgacctcttaggctcaaacaattctcccacttcagcctcccgagtagctggaactacaggcactcatcaccatgcctgactaattttttttttgtaaagactgtgtctcactatgttgcccagactggtcttgagctcctagactcaagcgatccttccacctcagtctcacaaattgctgggattacaggcatgagccacgcacatggtagaaactgaattattataagaaaaattggccggacccggtggctcacgcctgtaatcccaacactttggaaggccaaggcaggtggatc

SEQ ID NO: 2 - protein, 328 aa, Homo sapiens
MEDPPDTQFYVGSSLPQAGPWRVSAPPSGPPQFPAVVPGPSLEVARAHMLALGPQQLLAQDEEGDTLLHLFAARGLRWAAYAAAEVLQVYRRLDIREHKGKTPLLVAAAANQPLIVEDLLNLGAEPNAADHQGRSVLHVAATYGLPGVLLAVLNSGVQVDLEARDFEGLTPLHTAILALNVAMRPSDLCPRVLSTQARDRLDCVHMLLQMGANHTSQEIKSNKTVLHLAVQAANPTLVQLLLELPRGDLRTFVNMKAHGNTALHMAAALPPGPAQEAIVRHLLAAGADPTLRNLENEQPVHLLRPGPGPEDLRQLLKRSRVAPPGLSS

SEQ ID NO: 3 - DNA, 2046 bp, Unknown
  mouse
ggcacgagcccacatgcactcccagccacccaccgctgccaccgaggctgcattcgggtgacttaaacattcctgatgacttccttctgtcttcccacacacagtgagtcggcctgagcgtagtcactgcccaacacagacggtaaagaagcttctggaagaacagaggcgccgacagcagcagccagaggctgctggggtgccgggacacttctcccctcctctggtccagccactgactccatctgtgaatgaggcagagccaggccatactcatttccctgccttccaagagactgtggattccggacctggcaacctggctcccccttcaggctttgcagactgggaccctagcatccatgctacctatacagagggctctttcccattctctgcttctgaaggcttcctgactcctgatttctaccctccgtcagacccaggccggtcaggcccatttcccctggtgatggaggactctctggatacccggctgtatcctgagccttccctgtcacaggtagggtcctggagagtctctagtctcccctcaggatccccacagttgccttcacccaccggaccgtccctggagacagcccgagctcacatattggctctggggccccaacaactgctggcccaggatgaggaaggagacacgctcctgcacctgtttgctgcccgggggctgcgctgggcagcatatgctgcggccgaggtgttacagatgtaccgacagctggatattcgtgaacataaaggcaagacgccgctcctggtggcagctgctgccaaccagccactgattgtggaagacctgctgagcctgggagcagagcctaacgccactgaccaccagggccgttccgtcttgcacgtggctgccacctatggactcccgggagtcctttcggctgtgtttaagtccggcattcaagtggatctggaagccagagacttcgaaggccttacccccctgcacacagccgtcctggccctcaacgctgctatgctcccagctagtgtgtgtccaaggatgcagaattcccaagcccgagacagactgacttgtgtgcagatgttactgcaaatgggtgccagtcatacaagccaggagatcaagagcaacaagaccattctgcacttggctgtacaggctgccaaccccacgctggttcagctgctcctgggactgccaaggggggacctgcgggcctttgttaatatgaaggcccatggcaacactgccctccacatggcagccgccctgccccctgggccgccccaagaggccattgtgcggcacctgttggcagccggagcagacccgacactgcgcaacctggagaacgaacagcctgttcacttgctgcgacccgggccgggccctgaggggctccggcagctgttgaagaggagccgcacggcacccccaggcttgtcctcttaggaccgaaacccagaacctggactgattttccagtccccaccgtcccgtgggacagtcagcgtatgctcatgtcgcagattcatgataatatatgtgtaatatcctgccattagggtcttacattaaaactccaaagtggcacgggggggaggtgagcagtctccaatatttggggtctgtgacacctcctccccctacaaggggctttctaggtgatttctgtgaaatcgagcccacttgattgtttctgtgaaacactctagagacctttaactttctcctacctggatcagtcatgatcaccccccccagactgtcgtaccctgaatccccattggagtttccatgggatttgaggcatctgagtacctttcggtcaggattcctccccaggacccacacccctgagatgttggaaaggccctctgagattaatgtgtcctgagtcctggctacatcatcatttagaacccaatgtctctgacgctagcattccccgtgattgttccccttggtctgtgtatccctgtagctaccaattaaactcaatttggcctcaaaaaaaaaaaaaaaaaaaaaaaaaaaaaaaa

SEQ ID NO: 4 - protein, 607 aa, Unknown
  mouse
MEDSLDTRLYPEPSLSQVGSWRVSSLPSGSPQLPSPTGPSLETARAHILALGPQQLLAQMEDDTYSLQGWRVSPSGPQPGPSLEARAHLALGPQQLLAQDEEGDTLLHLFAARGLRWAAYAAAEVLQMYRQLDIREHKGKTPLLVAAAANQPLIVEDLLDEEGDTLLHLFAARGLRWAAYAAAEVLQYRLDIREHKGKTPLLVAAAANQPLIVEDLLSLGAEPNATDHQGRSVLHVAATYGLPGVLSAVFKSGIQVDLEARDFEGLTPLHTAVLALNLGAEPNADHQGRSVLHVAATYGLPGVLAVSGQVDLEARDFEGLTPLHTALALNAAMLPASVCPRMQNSQARDRLTCVQMLLQMGASHTSQEIKSNKTILHLAVQAANPTLVQLAMPCPRQARDRLCVMLLQMGAHTSQEIKSNKTLHLAVQAANPTLVQLLLGLPRGDLRAFVNMKAHGNTALHMAAALPPGPPQEAIVRHLLAAGADPTLRNLENEQPVLLLPRGDLRFVNMKAHGNTALHMAAALPPGPQEAIVRHLLAAGADPTLRNLEGEQPVHLLRPGGPEGLRQLLKRSRTAPPGLSSHLLRPGGPEGLRQLLKRSRAPPGLSS

SEQ ID NO: 5 - protein, 6 aa, Artificial Sequence
  motif
DSGLXS

SEQ ID NO: 6 - DNA, 21 bp, Artificial Sequence
  oligonucleotide
cccctggtgatggaggactct

SEQ ID NO: 7 - DNA, 21 bp, Artificial Sequence
  oligonucleotide
agagtcctccatcaccagggg

SEQ ID NO: 8 - protein, 338 aa, Unknown
  Murine
EAVVGVHDVGSHSLGTSLSLQNIMGSPMNTTQLGKSFFQWQVEQEESKLANIPQDQFLARDGDGDTFLHIAVAQGRRALSYVLARKMNALHMLDIKEHNGQSAFQVAVAANQHLIVQDLVNLGAQVNTTDCWGRTPLHVCAEKGHSQVLQAIQKGAVRSNQFVDLEATNYDGLTPLHCAVVAHNAVVHELQRNRQSHSPEVQDLLLRNKSLVDTIKQLIQMGAAVEAKDRKSGRTALHLAAEEANLELIPLFLELPSCLSFVNAKAYNGNTALHVAASLQYRVTQLDAVRLLMRKGADPSTRNLENEQPVHLVPDGPVGEQIRRILKGKSIQQRAPPY

SEQ ID NO: 9 - protein, 248 aa, Homo sapiens
MATRADEDGDTPLHIAVVQGNLPAVHRLVNLFQQGGRELDIYNNLRQTPLHLAVITTLPSVVRLLVTAGASPMALDRHGQTAAHLACEHRSPTCLPALLDSAAPGTLDLEARNYDGLTALHVAVNTECQETVQLLLERGADIDAVDIKSGRSPLIHAVENNSLSMVQLLLQHGANVNAQMYSGSSALHSASGRGLLPLVRTLVRSGADSSLKNCHNDTPLMVARSRRVIDILRGKATRPASTSQPDPS

SEQ ID NO: 10 - protein, 239 aa, Unknown
  Murine
TADARALLAGQRHLLMAQDENGDTPLHLAIIHGQTGVIEQIAHVIYHAQYLGVINLTNHLHQTPLHLAVITGQTRVVSFLLQVGADPTLLDRHGDSALHLALRAGAAAPELLQALLRSGAHAVPQILHMPDPEGLYPVHLAVHARSPECLDLLVDCGAEVEAPERQGGRTALHLATEMEELGLVTHLVTKLHANVNARTFAGNTPLHLAAGLGSPTLTRLLLKAGADIHAENEEPLCPL

SEQ ID NO: 11 - protein, 235 aa, Unknown
  Murine
TGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLQAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDL

SEQ ID NO: 12 - protein, 222 aa, Unknown
  Murine
WKQQLTEDGDSFLHLAIIHEEKALTMEVIRQVKGDLAFLNEQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQITWGRPSTRIQQQLGQLTLENLQMLPESEDEE